(19) United States
(12) Patent Application Publication
(10) Pub. No.: US 2003/0224403 A1
(43) Pub. Date: Dec. 4, 2003

(54) Lethal toxin cytopathogenicity and novel approaches to anthrax treatment

(76) Inventors: Serguei G. Popov, Bristow, VA (US); Edith Grene Carron, Leonardtown, MD (US); Jennifer Cardwell, Manassas, VA (US); Taissia Popova, Bristow, VA (US); Frank Klotz, Manassas, VA (US); Ken Alibek, Bristow, VA (US)

Correspondence Address: FINNEGAN, HENDERSON, FARABOW,, GARRETT & DUNNER, L.L.P., 1300 I Street, NW, Washington, DC, 20005-3315, US

(21) Appl. No.: 10/374,514

(22) Filed: Feb. 27, 2003

Related U.S. Application Data
(60) Provisional application No. 60/359,690, filed on Feb. 27, 2002.
(60) Provisional application No. 60/367,731, filed on Mar. 28, 2002.
(60) Provisional application No. 60/384,110, filed on May 31, 2002.
(60) Provisional application No. 60/390,111, filed on Jun. 21, 2002.
(60) Provisional application No. 60/429,357, filed on Nov. 27, 2002.

(51) Int. Cl. C12Q 1/68

(52) U.S. Cl. 435/6

(57) ABSTRACT

Inhibition of LeTx activity is provided as a treatment of anthrax infection. In particular, inhibition of the apoptotic effects of LeTx is provided as a targeted means of specifically treating anthrax infection. Treatments include inhibition of the Fas/FasL signaling pathway, inhibition of the effects of sFasL, inhibition of proteases of the caspase family and protection from loss of mitochondrial transmembrane potential in infected cells. Additionally, treatments targeting inhibition of apoptosis induced by LeTx activity include enhancement of the ERK (MAPK)-signaling pathway by agents including GM-CSF. The method of treating an infectious disease also comprises administering a combination of an antitoxin substance, which protects host cells from microbial toxin, and an antibiotic to an infected person. The anti-toxin substance includes different apoptosis inhibitors. Infection against which the treatment of the invention are effective include any disease leading to apoptosis of host cells such as, but not limited to, anthrax, plague, Ebola, or Marburg.

This application claims the benefit of priority of U.S. Provisional Application 60/359,690, filed Feb. 27, 2002 (attorney docket no. 08675-6006); U.S. Provisional Application 60/367,731, filed Mar. 28, 2002 (attorney docket no. 08675-6009); U.S. Provisional Application 60/384,110, filed May 31, 2002 (attorney docket no. 08675-6022); U.S. Provisional Application 60/390,111, filed Jun. 21, 2002 (attorney docket no. 08675-6027); and, U.S. Provisional Application 60/429,357, filed Nov. 27, 2002 (attorney docket no. 08675-6033), each of which are each incorporated by reference.

This invention was made with Government support under DAMD17-01-C-0033 awarded by U.S. Army Medical and Research Command. The Government has certain rights in the invention.

### BACKGROUND OF THE INVENTION

This invention relates to methods of treating infections of B. anthracis where the methods of treatment target the activity of LeTx. These methods also relate to methods of treating other infectious diseases such as, but not limited to, plague, Ebola, and Marburg.

Currently, there is no effective treatment for inhalational anthrax, the form most likely to be seen in a biological attack, except for the administration of antibiotics shortly after exposure to anthrax spores (Inglesby et al., 1999). As illustrated by the recent anthrax attacks in the United States, administration of antibiotics during the nonspecific, prodromal period can lead to increased survival, (Jernigan et al., 2001). However, side effects of antibiotics prohibit their prophylactic and post-exposure use for a large number of people belonging to different medical risk groups. Furthermore, by the time specific symptoms appear it is often too late for antibiotics to be effective. It is also conceivable that antibiotic resistant strains of anthrax could be used in possible future attacks. Therefore, there is a need in the art for supplements to traditional antibiotic intervention with new, safe, and efficient drugs that can be administered long-term.

In addition to anthrax, other infectious diseases, including but not limited to, plague, Ebola, and Marburg, present threats as terrorist weapons. There exists a need in the art to develop safe and effective treatments for all of these potential dangers.

Anthrax is a historically important model for understanding infectious diseases. The causative agent, Bacillus anthracis, is a Gram-positive, spore-forming organism, which generally infects herbivores (Hanna, 1998). Self-limiting infections of humans most often result from the introduction of spores through lesions in the skin, though the highly lethal form of anthrax is caused by inhalation of spores (LD₅₀≈10,000 spore particles).

In inhalational anthrax, inhaled anthrax spores are engulfed by alveolar macrophages that carry the spores to the mediastinal lymph nodes. The spores germinate inside the migrating macrophages, producing an antiphagocytic capsule and two toxins, lethal toxin (LeTx) and edema toxin (EdTx). Lysis of the macrophages allows release and proliferation of the bacteria in the lymphatic system, reaching concentrations of 10⁴-10⁵ bacteria per milliliter of lymphatic tissue (Guidi-Rontani et al., 1999a). The bacteria then escape into the bloodstream and continue to proliferate (Burgasov et al. 180). Bacteremia rises steadily until the last few hours before death, reaching 10⁸-10⁹ bacteria/ml (Hanna et al., 1993).

Death is attributed to severe respiratory distress and multi-system organ failure caused by sepsis and septic shock resulting from the overproduction of proinflammatory cytokines and other mediators, including stress hormones, small molecule and peptide neurotransmitters, etc. (Hanna et al., 1993; Hanna et al., 1994, Hanna, 1998). These conditions are poorly understood and are generally assumed to result from the overproduction of proinflammatory cytokines and other mediators (stress hormones, small molecule and peptide neurotransmitters, etc.).

The virulence of anthrax is determined mainly by its toxins, especially LeTx, which have been the subject of the majority of research on anthrax treatment and prevention. One theory of anthrax disease centers around LeTx function in late infection when toxin released into the bloodstream is implicated in the development of anthrax sepsis, septic shock, and death. This “extracellular” model of LeTx action suggests that LeTx attacks sensitive cells by binding to a putative cell surface receptor (Bradley et al. 2001) and ultimately translocating the lethal factor toxin subunit into the cell cytosol. The observation that binding of the protective antigen (PA) of anthrax is accompanied by a concomitant endocytosis of the LF subunit into the cell cytosol may support this theory (Singh et al., 1989; Singh et al., 1999). The theory is based on the assumption that circulating vegetative bacteria are the major source of secreted toxin, especially late in infection (Smith et al. 1955; Pezard et al., 1991), as modeled by intravenous injection of toxin into animals (Ezzell et al., 1984).

However, it has recently been observed that the toxin genes and their trans activator, atxA, are expressed within the macrophage early in infection, immediately after spore germination (Guidi-Rontani et al., 1999b). Recent studies (Pellizzari et al., 1999; Erwin et al., 2001) have suggested that LeTx-caused overproduction of proinflammatory cytokines and reactive oxygen species (Hanna et al., 1994) does not take place. In addition, LeTx-treated macrophages do not induce the release of IL-1β and TNF-α and are unable to respond to the stimulation with innate antigens, such as bacterial cell wall.

As an alternative theory, apoptosis is not uncommon in bacterial infections (Weinrauch et al., 1999; Gao et al., 2000 a,b). Certain pathogens have developed elegant mechanisms to modulate the fate of the host cell. However, in the case of anthrax, the action of LeTx has not been previously realized to be apoptotic, but instead was considered to be cytolytic (Hanna et al., 1993). Evidence that is consistent with the role of apoptosis, such as involvement of proteasome in LeTx activity (Tang et al., 1999), was previously rejected. Lin et al. (1996) observed apoptotic J774.A1 cells in the presence of LeTx after preincubation with calyculin A, and concluded that LeTx cannot mediate apoptosis in physiological conditions. Grinberg et al. (2001) found morphologically typical apoptotic lymphocytolysis after pathological analysis of the documented cases of anthrax from the Sverdlovsk epidemic.

It has been previously shown that Yersinia protein YopJ/P is able to decrease the production of TNF-α, and thus promote apoptosis of infected cells (Palmer et al., 1999). In addition, YopJ/P binds directly to the proteins of the MAPKK family and the binding blocks their activation. This pro-apoptotic Yersinia strategy is similar to LeTx-induced inhibition of the TNF-α production (Pellizzari et al., 1999; Erwin et al., 2001) and proteolytic inactivation of MAPKKs (Vitale et al., 1998; Pellizzari et al., 1999; Duesbury and Vande Woude, 1998). In both cases, cell death is induced by inhibiting of survival pathways rather than by directly triggering cell death. Therefore, in both Yersinia and anthrax infections, elimination of phagocytic cells through apoptosis and downregulation of inflammatory cytokines contribute to bacterial dissemination and disease progression.

Macrophages seem to play a central role in LeTx activity, because mice depleted of macrophages are resistant to lethal doses of toxin (Hanna et al., 1993, Hanna et al., 1994, Hanna, 1998). It has been demonstrated that the toxin can enter most cell types, but only certain macrophages and macrophage-like cell lines are susceptible to cytolysis by the toxin (Singh et al., 1989). Despite this extracellular theory of LeTx activity, the mechanism of LeTx intracellular activity remains largely unknown, although several approaches have been previously suggested to inhibit toxin intracellular activity, as well as to block its entry into susceptible cells (Sellman et al. 2001).

Neutrophils, monocytes, and tissue-based macrophages are major cellular components of the innate immune system, which represents the initial line of host defense against invading pathogens. Four cytokines, granulocyte colony-stimulating factor (G-CSF), GM-CSF, macrophage colony-stimulating factor (M-CSF), and interferon-gamma (IFN-γ), have received increasing attention as potential adjunctive immunomodulatory agents for treatment of infectious diseases. In vitro and in vivo studies have shown that these cytokines can augment the functional antimicrobial activities of neutrophils. Similarly, GM-CSF, M-CSF, and IFN-γup-regulate multiple antimicrobial mechanisms in monocytes and macrophages. Studies in animal models have shown the potential use of each of these cytokines for the treatment of infections caused by a variety of bacterial, fungal, and parasitic diseases. However, clinical experience with these immunomodulatory cytokines is relatively limited, and controlled clinical trials are necessary to define specific indications for the administration of these cytokines in therapeutic regimens (for reviews see Liles, 2001; Armitage, 1998).

Clinical indications for use of recombinant human GM-CSF have expanded considerably since the drug first became available in the early 1990s for acceleration of myeloid engraftment in neutropenic patients. Initial clinical trials of GM-CSF were based on prevailing knowledge of the biologic effects of endogenous GM-CSF at the time and therefore concentrated on the drug's myeloproliferative effects in myelosuppressed patients. As additional information accumulated from in vitro research and from results of clinical trials, it became apparent that GM-CSF had diverse biologic effects and played a vital role in various functions of the immune system, including responses to inflammation and infection, as well as in hematopoiesis. Consequently, a variety of potential clinical uses for GM-CSF are under investigation, such as prophylaxis or adjunctive treatment of infection in high-risk settings or immunosuppressed patient populations, use as a vaccine adjuvant, and use as immunotherapy for malignancies.

Stimulation of murine macrophages with TNF-α, IFN-γ, and GM-CSF, but not M-CSF, was associated with mycobacteriostatic and/or mycobactericidal activity in macrophages. Treatment with these cytokines at 24 h prior to infection with mycobacteria was considerably more effective than treatment after the beginning of infection (Hsu et al., 1995). In another study, treatment of murine macrophages with murine GM-CSF for 24 h enhanced their capacity to restrict growth of C. albicans (Yamamoto et al., 1997). A combination of GM-CSF with antibiotics (amikacin or azithromycin) was associated with a significant increase in killing of Mycobacterium both within cultured macrophages and in infected mice (Bermudez et al., 1994). In a small pilot study, human recombinant GM-CSF (Sargramostim®) appeared to exert a beneficial effect on the mucosal mycoflora and was suggested as a possible adjunctive therapy in the management of fluconazole-refractory mucosal candidiasis in advanced HIV-positive patients (Vazquez et al., 2000). Singh and Singh (2001) reported a significant suppression of the parasitaemia after co-administration of GM-CSF and met-enkephalin against blood-induced Plasmodium berghei infection in Swiss mice, apparently through macrophage-mediated mechanisms. GM-CSF in combination with appropriate antibiotics was found to be an effective and safe treatment for the management of patients with pneumonia and severe hematopoietic dysfunction (Dierfort et al., 1997). In Phase III trial subcutaneous injections of GM-CSF three times per week for 24 weeks significantly reduced the incidence of overall infections and delayed time to first infection in HIV patients (Angel et al., 2000).

The invention is based on a novel mechanism of LeTx-induced cell death in mouse and human cells. Elucidation of the bacterial molecules and the mechanisms by which B. anthracis trigger apoptosis of host cells provides valuable information for new approaches to disease treatment.

### SUMMARY OF THE INVENTION

This invention aids in fulfilling this need in the art by providing methods of treatment based on the mechanisms underlying proinflammatory response inhibition by LeTx. In general, the invention provides for an arsenal of antitoxin agents according to these treatments. The invention provides an antimicrobial treatment by inhibiting toxin intracellular activity through enhancement of the activity of infected macrophages. This aspect of the invention allows for a decrease in intracellular bacterial survival. In general, the inhibition of LeTx activity within macrophages is an early treatment of anthrax that prevents the initiation of infection.

Specifically, the invention provides for a method of treatment by inhibition of the Fas/FasL signaling pathway, inhibition of proteases of the caspase family, and protection from loss of mitochondrial transmembrane potential in infected cells. Additionally, treatments targeting inhibition of apoptosis induced by LeTx activity include enhancement of the ERK (MAPK)-signaling pathway by agents including GM-CSF. Other effectors of LeTx activity include IL-4, IL-6, and matrix metalloprotease inhibitors.

This invention also aids in fulfilling the needs in the art by a novel treatment for anthrax, as well as other infectious diseases such as, but not limited to plague, Ebola, and Marburg, in a combination therapy of inhibitors of lethal toxin-induced signaling and antibiotics. The lethal toxin-induced signaling inhibitors include, but are not limited to, caspase inhibitors.

### BRIEF DESCRIPTION OF THE DRAWINGS

This invention will be described in greater detail with reference to the drawings.

FIG. 1 depicts staining of LeTx treated RAW 264.7 cells with YO-PRO-1 (apoptosis-specific, green fluorescent) (A) and propidium iodide (PI; late apoptosis and necrosis-specific, red fluorescent) (B). The cells were treated with different concentrations of toxin for 4 h in culture media. Staining was analyzed by flow cytometry. The treatments are as follows: thick solid line, PA (500 ng/ml); thin solid line, PA (500 ng/ml)+LF (4 ng/ml); dashed line, PA (500 ng/ml)+LF (8 ng/ml); gray area, untreated control cells.

FIG. 2 depicts staining of LeTx treated human peripheral blood monocytes preincubated for 48 h with IFN-γ (100 u/ml). The cells were treated with different concentrations of toxin for 4 h in culture media with (A) or without 10% FCS (B). Cells were analyzed by flow cytometry after staining with YO-PRO-1 using a gate region corresponding to necrotic and apoptotic cells. Thick line, PA (500 ng/ml)+LF (500 ng/ml); thin line, PA (500 ng/ml); dotted line, staurosporine (50 μM). Gray area, untreated cells.

FIG. 3 depicts the Tunel assay of nucleosomal degradation of RAW 264.7 cells in presence of LeTx (16 ng/ml). Cells were treated for 4 h with: LeTx (solid thick line), 500 ng/ml PA alone (gray area), or staurosporine (5 μM) as a positive control (dashed line). Fluorescence in channel 1 was recorded, and the population of apoptotic cells was gated using a positive control as reference.

FIG. 4 depicts flow cytometry analysis of RAW 264.7 cells stained with a mitochondrial transmembrane potential-sensitive dye, JC-1. Histograms of fluorescence in channel 1 correspond to (from right to left): control cells without toxin, cells treated for 4 h with LeTx at concentrations 8, 16, and 32 μg/ml of LF in presence of 500 ng/ml of PA, and cells treated with staurosporine (5 μM) as a positive control.

FIG. 5 depicts the effect of mGM-CSF in delaying the death of RAW 264.7 cells induced by LeTx (16 ng/ml of LF, 500 ng/ml PA). Cells were prestimulated with mGM-CSF for 22 h, and LeTx was added for 4 h. Cells were stained and analyzed by flow cytometry as in FIG. 1. The amounts of cells in quadrants of dot-plots were counted. Cells are designated as: alive (both YO-PRO-1⁻ and PI⁻), apoptotic (both YO-PRO-1⁺, PI⁺ and PI⁻), and dead (YO-PRO-1⁻, PI⁺).

FIG. 6 depicts the effect of caspase inhibitors on the staining pattern of RAW 264.7 cells detected by flow cytometry. The cells were incubated with one of the caspase inhibitors (20 μM) for 15 h, then LeTx (4 ng/ml of LF+500 ng/ml PA) was added for 4 h, and cells were stained as described in FIG. 1 legend. The numbers above the histograms correspond to caspase numbers. Histograms of LeTx-treated and untreated cells are marked as LeTx and control, respectively.

FIG. 7 depicts the increase in Fas expression after stimulation with cytokines in RAW 264.7. Cells were stained with anti-Fas FITC-labeled anti-Fas antibody after stimulation with recombinant mouse cytokines (100 u/ml) for 24 h. Gray areas show unstimulated cells.

FIG. 8 depicts anti-FasL neutralizing antibody protection of RAW 264.7 cells from killing by LeTx. Cells were incubated with the indicated concentration of antibody and 4 ng/ml of LeTx for 15 h. Cell numbers were determined by flow cytometry as in FIG. 5.

FIG. 9 depicts the lack of inhibition of LeTx activity in RAW 264.7 cells by inhibitors of MAPKK 1/2 (A) and p38 (B). After 5 minutes of incubation with the inhibitor, LeTx (8, 32, and 64 ng/ml of LF in presence of 500 ng/ml PA) was added to cells for 4 h, and the viability of cells was assessed by MTT assay.

FIG. 10 depicts flow cytometry of RAW 264.7 cells (1×10⁶/ml) after infection with anthrax (Sterne) spores (10×10⁶/ml) at different times after staining as in FIG. 1. At the beginning of infection, spores are undetectable in scatter channels (A). Signals from growing bacterial cells (B) practically do not overlap with signals from uninfected (C), and infected (D) RAW 264.7 cells. Histograms (K to N) were gated to exclude signals from bacterial cells (B, gray dots), and correspond to dot-plots (E to F) above them.

FIG. 11 depicts the decrease in phagocytic capacity of RAW 264.7 cells in response to LeTx. The spores (2×10⁶/well) were added to cells (2×10⁵/well), and after 30 min incubation the cells were lysed, and the viability of remaining spores and vegetative bacteria was determined using the Alamar Blue® technique.

FIG. 12 depicts increases in bactericidal activity of murine RAW 264.7 cells (A) and human PBMCs (B) infected with anthrax (Sterne) spores in response to bestatin. Cells (2×10⁵/well) were incubated with bestatin for 1 h, then spores were added to cells for 3 h. After incubation, the cells were lysed, and the viability of remaining spores and vegetative bacteria was determined using the Alamar Blue® technique. The spore:cell ratio was 5:1 (filled bars, A), 10:1 (open bars, A), and 10:1 (B). PBMCs from two donors were used (B). Before addition of spores, the PBMCs were prestimulated with IFN-γ (100 u/ml) for 24 h.

FIG. 13 depicts the differential influence on LeTx activity in RAW cell viability. LeTx (100 ng/ml) was added to cell preincubation with indicated cytokines overnight. Viability of the cells was measured with the MTT assay. Cell viability relative to control cells treated with toxin only is presented.

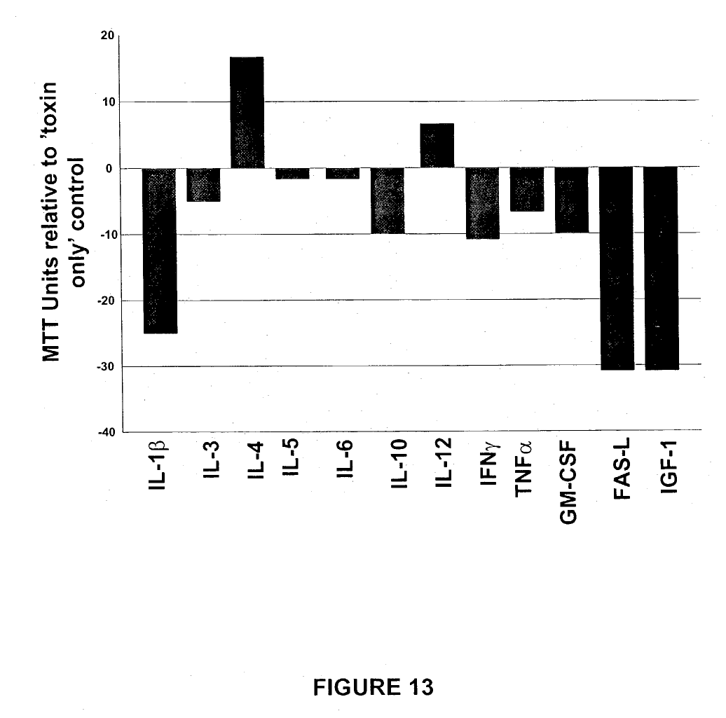

FIG. 14 depicts a Tunel assay of nucleosomal degradation of PBMCs in the presence of LeTx. Cells were preincubated with IFN-γ (100 u/ml) for 48 h and treated with different concentrations of toxin for 15 h in culture media. Staining was measured by flow cytometry. The different treatments are depicted as follows: dotted line, PA (500 ng/ml); thick solid line, PA (500 ng/ml)+LF (500 ng/ml); thin solid line, staurosporine (50 μM) as positive control; gray area, untreated control cells. Fluorescence in a green channel was recorded.

FIG. 15 depicts production of proinflammatory cytokines by human PBMCs activated by B. anthracis cell wall (CW) in presence of LeTx. Cytokine release was detected by ELISA after 48 h stimulation. The numbers refer to the following treatments: cells only control (1), CW,1 μg/ml (2), CW, 0.5 μg/ml (3), CW, 0.1 μg/ml (4), LF, 0.5 μg/ml+PA, 0.1 μg/ml (5), CW, 1 μg/ml+LF, 0.5 μg/ml+PA, 0.1 μg/ml (6), CW, 0.5 μg/ml+LF, 0.5 μg/ml+PA, 0.1 μg/ml (7), CW, 0.1 μg/ml+LF, 0.5 μg/ml+PA, 0.1 μg/ml (8), LF, 0.5 μg/ml (9), PA, 0.1 μg/ml (10), LPS, 10 ng/ml (11).

FIG. 16 depicts apoptosis resulting from anthrax infection as demonstrated in a histogram of green fluorescence of PBMCs in a flow cytometry experiment 24 h after infection with anthrax (Sterne) spores. PBMCs were gated using a scatter plot. Staining was performed as in FIG. 1. The spore:cell ratio was 10:1. Signals from growing bacterial cells do not overlap with signals from apoptotic cells.

FIG. 17 depicts that mGM-CSF delays necrotic changes in Raw264.7 cells induced by LeTx (32 ng/ml) at the late apoptotic step. Cells were prestimulated with mGM-CSF for 22 h, and LeTx was added for 4 h. Histogram plots were generated from flow cytometry data obtained after staining with YO-PRO-1 and PI as in FIG. 1. Gray areas: cells without toxin; solid lines: cells after incubation with toxin. The right panels correspond to stimulated cells.

FIG. 18 depicts mouse inflammatory cytokine RNA gene expression in Balb/c peritoneal macrophages. Lanes contain the multiplex PCR samples: 1) positive controls, 2) cells control, 3) cells challenged with CW, 4) cells challenged with LeTx, 5) cells challenged with CW and LeTx, 6) cells challenged with LPS, and 7) positive control. The fragments from the genes analyzed are indicated with arrows, from top to bottom: GADPH, IL-6, TNF-α, IL-1, TGF-β, GM-CSF.

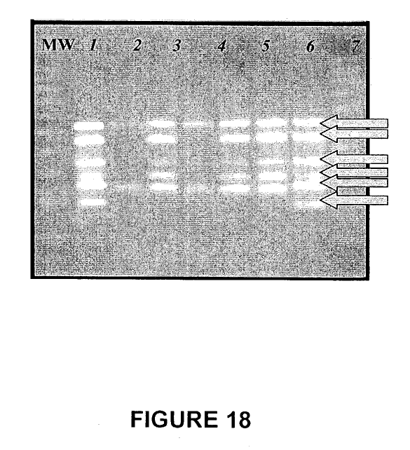

FIG. 19 depicts human donor (RC46) monocyte cDNA amplified from mRNA using primers for Fas. The lanes represent the following treatments: Lane 1, control monocytes; lane 2, 1 μg/ml of CW; lane 3, 100 ng/ml LF and 500 ng/ml PA; lane 4, 100 ng/ml LF, 500 ng/ml PA and 1 μg/ml of CW; lane 5, 100 ng/ml of PMA. All monocytes were challenged in serum-free media, and incubated for 4 h before RNA harvest.

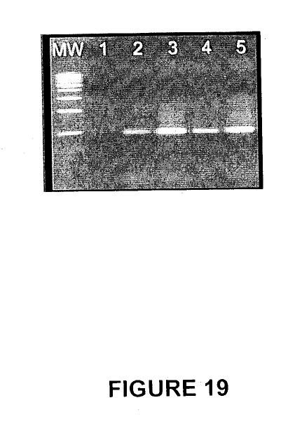

FIG. 20 depicts Fas and FasL RNA expression pattern in THP-1 cells. Lanes contain: 1) positive control, 2) positive control plus spike, 3) THP-1 cells control, 4) THP-1 cells with CW, 5) THP-1 cells with LeTx, 6) THP-1 cells with a pokeweed mitogen. Lanes 3-6 show Fas expression and lanes 7-10 show FasL expression in the same order as in lanes 3-6.

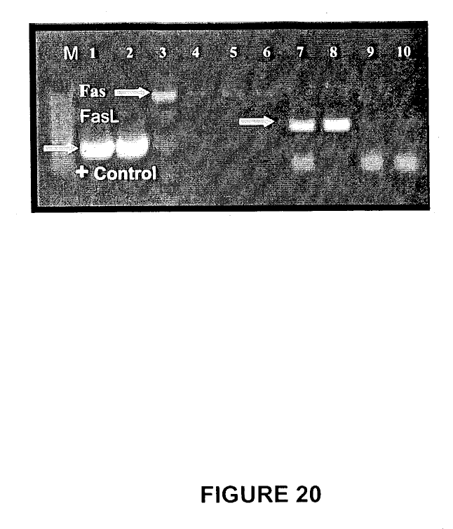

FIG. 21 depicts a 192-gene section of a 5,300 gene microarray chip hybridized with a control THP-1 cell sample and a THP-1 cell sample stimulated for 12 h with both LeTx (composed of 100 ng/ml PA, and 500 ng/ml LF) and CW.

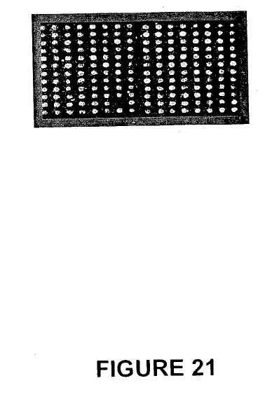

FIG. 22 depicts survival of inhibitor treated A/J mice infected with 5×10⁵ CFU of B. anthracis spores. Mice were treated at days −1, 0, 1 and 4 with ciprofloxacin (60 mg/kg), doxycycline (160 mg/kg), neomycin (160 mg/kg), chloroquine (40 mg/kg), verapamil (20 mg/kg), trypsin (0.5 mg/kg), bafilomycin A1 (0.025 mg/kg), bestatin (4 mg/kg), Z-VAD-fmk (20 mg/kg), or with a mock treatment.

FIG. 23 depicts survival of A/J mice that were injected peritoneally with 5×10⁵ spores per mouse (approximately 4×LD50) on day 0. Mouse GM-CSF (mGM-CSF) was administered intranasally on days −2, 0, +2, +4 at a dose of 2×10⁴ units/mouse/day.

FIG. 24 depicts survival of A/J mice that were injected peritoneally with 2×10⁵ and 5×10⁵ B. anthracis spores per mouse (approximately 2×LD50 and 4×LD50) on day 0. Z-VAD-fmk was administered intraperitoneally on days −2, −1, 0 at a dose of 20 mg/kg/day.

FIG. 25 depicts survival of A/J mice that were injected peritoneally with 2×10⁵ and 5×10⁵ B. anthracis spores per mouse (approximately 2×LD50 and 4×LD50) on day 0. Z-VAD-fmk was administered intraperitoneally on days 0, +1, +2 at a dose of 20 mg/kg/day.

FIG. 26 depicts survival of A/J mice that were injected intraperitoneally with 2×10⁵ and 5×10⁵ B. anthracis spores per mouse (approximately 2×LD50 and 4×LD50) on day 0. Bestatin and Z-VAD-fmk were administered intraperitoneally on days −1,0, +1, +4 at a dose of 5 mg/kg/day and 20 mg/kg/day, respectively.

FIG. 27 depicts survival of DBA/2 mice that were injected intraperitoneally with approximately LD90 of B. anthracis spores on day 0. Subcutaneous neomycin administration to two groups of mice started on day −1 and continued for 11 more days at the doses of 1 mg/kg/day and 5 mg/kg/day. Another group of mice received intraperitoneal ciprofloxacin treatment starting at day 1 for 10 days at the dose of 50 mg/kg/day. Finally, two additional groups received neomycin at either 1 or 5 mg/kg/day plus ciprofloxacin at 50 mg/kg/day.

FIG. 28 depicts survival of DBA mice that were injected intraperitoneally with approximately LD90 of B. anthracis spores on day 0. Bestatin was administered subcutaneously on days −1 at doses of 1 mg/kg/day and 5 mg/kg/day, The treatment was continued for 11 more days. Intraperitoneal ciprofloxacin treatment started at day 1 for 10 days at a dose of 50 mg/kg/day. Additional groups received one of two combinations of bestatin (5 mg/kg/day or 25 mg/kg/day) plus ciprofloxacin (50 mg/kg/day).

FIG. 29 depicts survival A/J mice that were injected intraperitoneally with B. anthracis spores (1×10⁴ CFU) on day 0. Ciprofloxacin was administered intraperitoneally at a dose of 60 mg/kg. Administration of ciprofloxacin began on different days, as indicated, and continued for 5 days. Treatment was administered once daily.

### DETAILED DESCRIPTION OF THE EMBODIMENTS

The invention provides a method of treatment of anthrax infection by targeting the activity of LeTx.

Macrophages have long been implicated in the development of inhalational anthrax. In the process of anti-microbial response the alveolar macrophages engulf the inhaled spores and carry them to mediastinal lymph nodes, where a cascade of intracellular reactions becomes induced to eliminate pathogen. However, a lethal dose of spores can overwhelm the bactericidal capacity of macrophages. As a result, some spores survive and germinate within the macrophages producing an antiphagocytic capsule and two toxins, LeTx and edema toxin (EdTx). Escape of bacilli from infected cells allows release and proliferation of the bacteria in the lymphatic system and consequent development of systemic disease. (Guidi-Rontani et al. 1999.)

Macrophages were suggested as the major mediators of LeTx activity because it has been shown that mice depleted of macrophages are resistant to lethal doses of toxin. (Hanna et al. 1993; Hanna et al. 1994; Hanna, 1998.) However, the toxicity of LeTx activity in vitro has been difficult to demonstrate. Experiments with animals show that the strains of mice sensitive to toxin are still relatively resistant to anthrax infection. (Welkos et al. 1986; Kline et al. 1963.) Even though both humans and rodents are susceptible species, no correlation has been found between LeTx toxicity in vitro and the infectious process in vivo. Cytolysis after LeTx treatment was reported only in a few situations, such as in peritoneal exudate macrophages from C3H/HeNHsd mice (Friedlander 1986), and in the murine macrophage cell lines, RAW 264.7 and J774.A1. (Hanna et al. 1993; Singh et al. 1989.) The intracellular enzymatic activity of LeTx toward MAPKKs (17-20) does not correlate with the resistance. No cytopathic effect of LeTx was reported for human white blood cells, indicating that cell lysis was not a biologically relevant effect of LeTx.

LeTx-induced cell death contributes significantly to the pathology of anthrax. (Popov et al., 2002a) Furthermore, LeTx causes macrophage death by inducing apoptosis, but not with a cytokine burst and corresponding oxidative lysis. A previously unrecognized function of LeTx consists of the suppression of the antimicrobial function of host macrophages. In addition to its role as exotoxin, lethal toxin can be considered as an early intracellular virulence factor secreted by vegetating bacilli inside macrophages within a few hours after the spore entry into the host. In the early stages of disease LeTx becomes active inside the infected macrophage promoting both intracellular bacterial replication and macrophage apoptosis (Popov et al., 2002 a, b).

The invention provides for identification and characterization of major apoptotic systems involved in lethal toxin action. These apoptotic systems leading to LeTx-induced cell death include Fas (CD95)/FasL and, potentially, TRAILRs/TRAIL (Apo-2). These systems are characterized with flow cytometry, Western blotting, ELISA and RT-PCR, as well as specific neutralizing antibodies. Susceptibility of murine macrophage cell lines, peritoneal macrophages, human peripheral blood white cells, monocytes/macrophages and neutrophils are demonstrated.

The Fas/FasL system is involved in LeTx-induced macrophage death. This finding has several important implications and opens new avenues for therapeutic interventions. Apoptotic macrophages are capable of fast release of toxic soluble mediators, such as soluble FasL (Kiener et al., 1997), and of spreading death to bystander cells (Brown and Savill, 1999). Such signaling in response to LeTx provides a mechanism for amplifying the initial effect of toxin. Soluble FasL is toxic at low doses (Tanaka et al., 1997) and can cause different pathological conditions relevant to anthrax, such as acute respiratory distress syndrome (Matute-Bello et al., 1999; Matute-Bello et al., 2001; Serrao et al., 2001), systemic tissue injury (Tanaka et al., 1995), and neurotoxicity (Fish et al., 1968; Chiarugi et al., 2001).

The role of soluble apoptotic mediators, such as sFasL, released by intoxicated cells is demonstrated by conditioned media transfer. The ability of these mediators from LeTx-induced apoptosis in macrophages to cause death of the surrounding cells is demonstrated. The apoptotic death of B. anthracis-infected macrophages is demonstrated in comparison with LeTx-treated cells.

In another embodiment, the invention provides for inhibition of apoptosis as a method of treating anthrax infection. In yet another embodiment, apoptosis is inhibited by interfering with a chain of intracellular signaling events involving Fas ligand (FasL), which is known as an inducer of cell apoptosis. The invention provides for antitoxin agents that target the Fas-mediated apoptotic signaling pathway.

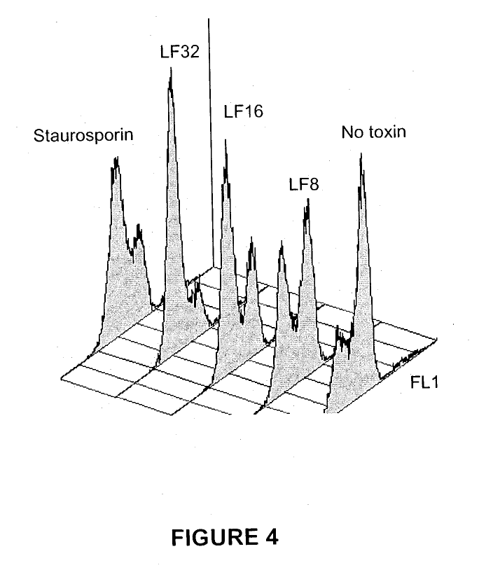

In addition, LeTx initiates activation of a number-of caspases, including the initiator caspase-3, and the effector caspase-8, an effect that correlates with a current view on apoptosis (Gao et al., 2000 a, b; Gublins et al., 2000). Prevention of LeTx-induced cytotoxicity by anti-FasL neutralizing antibodies (FIG. 4) indicates a significant role for Fas/FasL interaction in this process. This is consistent with the observation that proteases of the caspase family, especially caspases-1 (ICE), -3, -4, and -8, are implicated in Fas-mediated apoptosis (Kamada et al., 1997).

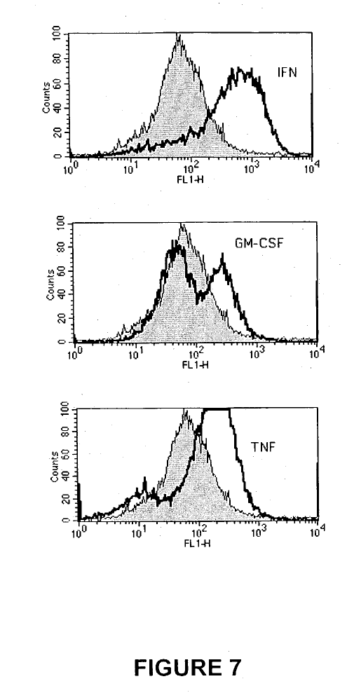

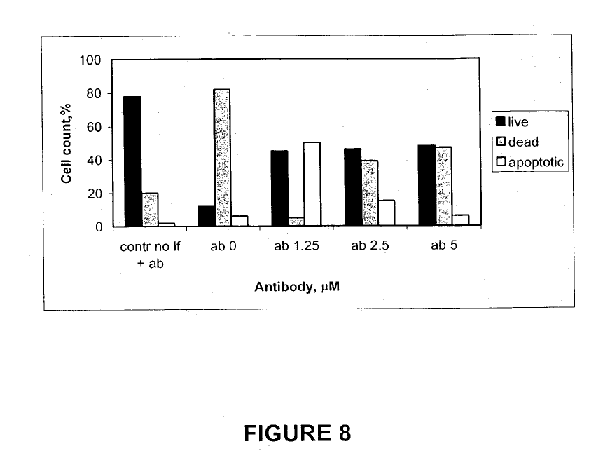

Apoptotic events in LeTx-treated cells are initiated by Fas/FasL interaction on the cell surface. Both mouse and human cells expressed high level of surface bound Fas (FIG. 7). Their increased expression in the presence of IFN-γ correlated with sensitization of the cells to Fas-mediated apoptosis (Ossina et al., 1997). The prevention of LeTx-induced cytotoxicity by anti-FasL neutralizing antibodies (FIG. 8) indicates a significant role of Fas/FasL interaction in this process.

The invention also provides for transcriptional profiling of lethal toxin activity and anthrax infection in the host cells. The expression array technique is used to identify the expression pattern of host genes relevant to signaling induced by lethal toxin and anthrax infection focusing mainly on the apoptotic pathways. RT-PCR provides more detailed information on the intensity and the time course of response.

Comparisons between (1) toxin-treated versus nontreated cells (murine macrophages and human peripheral blood cells); (2) B. anthracis (Sterne)—and delta-Sterne (pXO1⁻)—infected versus uninfected cells; (3) animals challenged with the spores of the above strains of B. anthracis, demonstrate the expression patterns of host genes. In addition, circulating blood cells and spleen cells of infected animals are also used to show expression patterns. These expression patterns provide a broader picture of cellular transcriptional activity in normal and disease conditions. Similarities and differences in the spectrum, intensity and the time course of cellular responses to the lethal toxin versus the intracellular infectious process define the relative contribution of the toxin in the pathogenesis.

The invention provides for unique information that demonstrates particular signaling pathways as potential pharmacological targets for anthrax prophylaxis and treatment. Inhibitors of these selected pathways are used in cell culture and in mice challenged with anthrax spores to demonstrate their protective effect.

Apoptosis is usually regarded as a slow process, but it has been shown that in certain systems it can proceed very fast, taking only several minutes (Fladmark et al., 1999; Hohlbaum et al., 2001). This seems to be the case with Raw 264.7 cells which are extremely sensitive to LeTx. Therefore, both necrosis and apoptosis may occur. Many, bacterial pore-forming toxins can induce both necrosis and apoptosis (Weinrauch et al., 1999). Several reports (Tsujimoto et al., 1997; Nicotera et al., 1997; Nicotera et al., 1999) have indicated that these two processes can take place simultaneously in tissues or cell cultures exposed to the same stimulus.

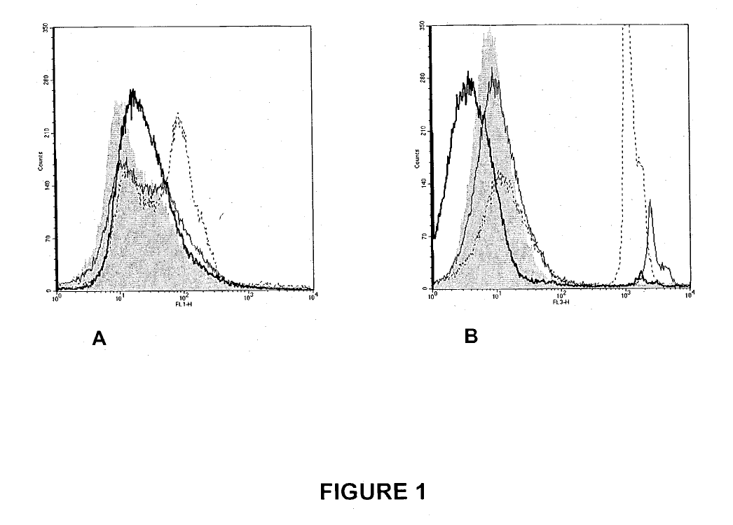

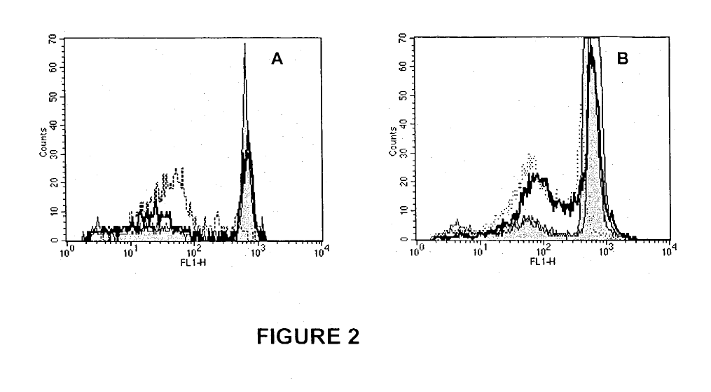

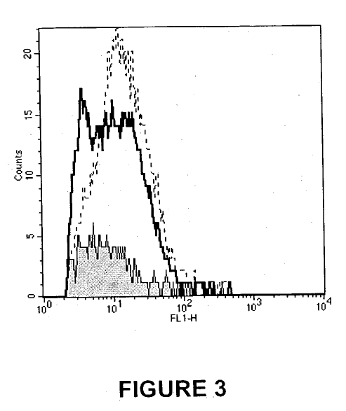

Whether cells die by necrosis or apoptosis is thought to depend largely on the severity of the insult (Ankarcrona et al., 1995; Bonfoco et al., 1995). For example, the induction of either apoptosis or necrosis appears to be dependent on concentration of S. aureus α-toxin (Jonas et al., 1994). This may explain why the necrotic-like death prevailed over the apoptotic death component of Raw 264.7 cells in experiments at high cytolytic concentrations of the toxin described in the literature. Low, non-lytic concentrations of LeTx better reflect initial process in infected macrophages, whereas high concentrations of the toxin in blood could be detected only late in the infection process (Smith et al., 1955). Indeed, the infection of macrophages with anthrax (Sterne) spores showed a large population of cells with membrane staining patterns typical for apoptosis (FIG. 1 and 2). It is difficult to detect DNA oligomerization in intoxicated Raw 264.7 cells using an agarose gel technique (data no shown); however, the more sensitive Tunel assay confirms the DNA fragmentation (FIG. 3). Several laboratories have shown that the early morphological changes of nuclear chromatin coincide with the appearance of high molecular weight fragments, while the formation of the DNA ladder is a rather late event, occurring during or after apoptotic body formation has taken place (Cohen et al., 1994; Bicknell et al., 1995; Walker et al., 1997).

LeTx is considered to be a major anthrax virulence factor. However, there are only a limited number of available experimental in vitro systems where its cytolytic activity has been demonstrated. The Raw 264.7, as well as J774.A1 mouse macrophage-like cell lines, are among the most susceptible, while the majority of other cells, including human white blood cells, are resistant (Fedotova et al., 1970; Friedlander, 1986). The nature of cell resistance to the toxin remains unknown. Recent data demonstrate the involvement of Kif1C, a kinesin-like motor protein, late in the process of the toxin action (Watters et al., 2001). However, the intracellular targets of LeTx enzymatic activity, known as MAPKKs (Pellizzari et al., 1999; Pellizzari et al., 2000), undergo proteolysis with the same rate in sensitive and resistant cells. Cytokine burst was previously implicated in the mechanism of LeTx-induced cell lysis, though recent data argue with this point of view (Pellizzari et al., 1999; Erwin et al., 2001).

In contrast to Raw 264.7 cells, and in agreement with previous reports (Fedotova et al., 1970; Friedlander, 1986), human PBMCs are almost completely resistant to LeTx (FIG. 2). Apoptosis and proliferation may be viewed in terms of a “growth equation,” with too much growth signal resulting in little death (Fadell et al., 1999). In human PBMCs, a strong continuous stimulus suppresses the death signaling pathway, so that LeTx is not able to initiate apoptosis.

It has been shown that RAW 264.7 cells infected with anthrax undergo changes typical of apoptotic death at concentrations lower than required for lysis. (Popov et al. 2002a.) Cellular membrane apoptotic changes in cells infected with anthrax spores were also detected. In these experiments, infected human monocyte-derive macrophages behave similarly to the murine cells. The finding that a LeTx inhibitor, bestatin, protects infected cells, further supports a correlation between LeTx-induced apoptosis and the impairment in macrophage function.

The murine macrophage-like cell lines, RAW 264.7 and J774.A1 are among the most susceptible to the cytolysis by LeTx, while a majority of other cells, including human white blood cells, are resistant (Friedlander, 1986). Both humans and rodents are susceptible species, however, no correlates were found between LeTx activity detectable in vitro and the infectious process. Anthrax infection in both mouse and human phagocytes leads to the reduction in their bactericidal capacity against spores and germinating bacilli, and the appearance of apoptotic cells. Apoptosis changes can also be found in LeTx-treated human PBMCs and monocytes. The conditions of serum withdrawal in presence of IFN-γ substantially increase susceptibility of PBMCs to LeTx (FIG. 2), consistent with Fas-mediated apoptosis (Ahn et al., 2001). The apoptotic changes in PMMs explain the ability of LeTx to reduce production of proinflammatory cytokines by cells stimulated through innate immune receptors. Macrophage inactivation, but not direct killing, contributes to bacterial dissemination and disease progression. A proinflammatory status of macrophages rather than cytolysis is a marker of cell susceptibility to LeTx.

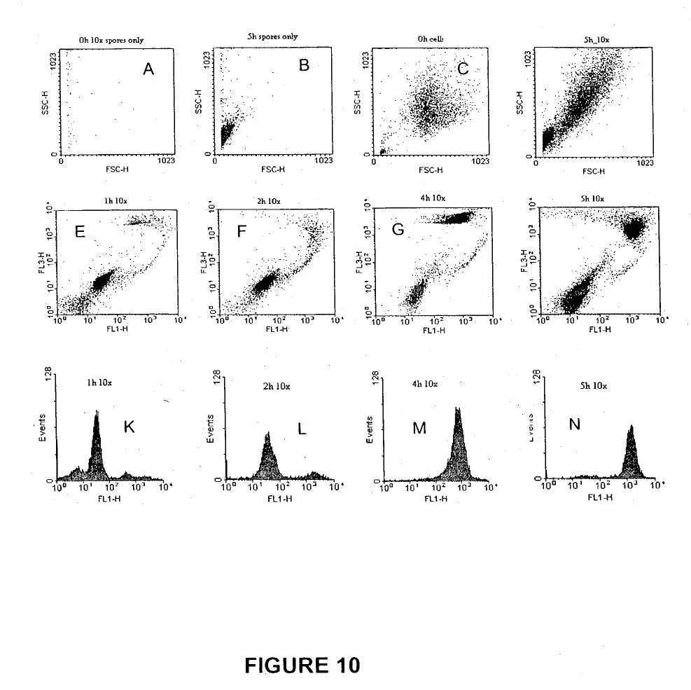

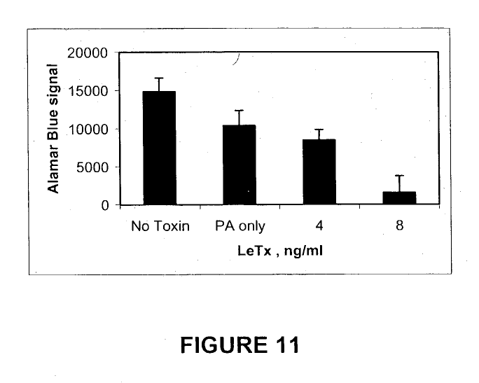

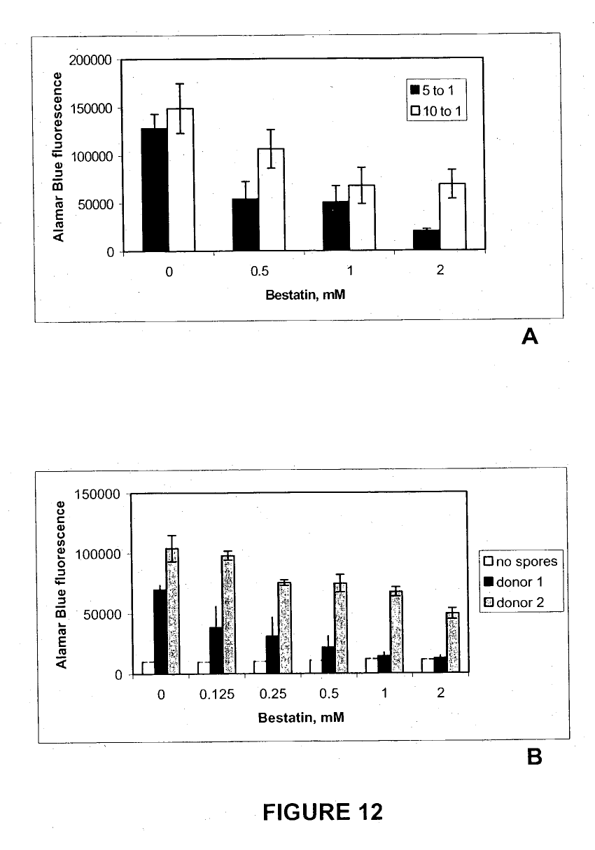

As shown, LeTx-treated Raw 264.7 cells decreased their phagocytic capacity indicating a functional impairment of infected macrophages (FIG. 11). In addition, bestatin, a known inhibitor of LeTx activity (Menard et al., 1996), restores a bactericidal activity of macrophages infected with anthrax spores both in case of mouse Raw 264.7 cells and human PBMCs. (FIG. 12) Finally, the process of intracellular macrophage infection is shown to lead to the appearance of apoptotic cells (FIG. 10), as anticipated from the pro-apoptotic LeTx function, and the early expression of toxin genes in macrophages infected with anthrax spores (Guidi-Rontani, 1999).

Mouse Raw 264.7 cells exposed to LeTx undergo changes in membrane permeability, DNA fragmentation, and mitochondrial membrane potential that are typical for apoptosis (Rathmell et al., 1999; Gao et al., 2000 a, b; Gublins et al., 2000, Popov 2002a). While it is difficult to detect DNA oligomerization in intoxicated Raw 264.7 cells using an agarose gel technique (data no shown); however, the more sensitive Tunel assay confirms the DNA fragmentation (FIG. 3). Several laboratories have shown that in generally in apoptosis early morphological changes of nuclear chromatin coincide with the appearance of high molecular weight fragments, while the formation of the DNA ladder is a rather late event, occurring during or after apoptotic body formation has taken place (Cohen et al., 1994; Bicknell et al., 1995; Walker et al., 1997).

In addition, LeTx initiates activation of a number of caspases, including the initiator caspase-3, and the effector caspase-8, an effect that correlates with a current view on apoptosis (Gao et al., 2000a, b; Gublins et al., 2000).

Embodiments of this invention are based on the role of signaling pathways that are involved in apoptosis. Conditions of serum starvation are known to increase Fas-mediated apoptosis in human diploid fibroblasts (Ahn et al., 2001), and to activate stress-responsive JNK pathway in neuronal cells (Le-Niculescu et al., 1999). In the experiments shown in Example 6, IFN-γ was used to differentiate monocytes into monocyte-derived macrophages (MDMs). Serum withdrawal in the presence of IFN-γ substantially increased PBMC's susceptibility to LeTx (FIG. 12).

The apoptotic changes in MDMs explain the ability of LeTx to reduce production of proinflammatory cytokines by cells stimulated through innate immune receptors (Akira et al., 2001). Macrophage inactivation, but not direct killing, can contribute to bacterial dissemination and disease progression. The fact that bestatin, the inhibitor of LeTx, reduces bacterial burden in infected MDMs implicates LeTx as an early intracellular virulence factor secreted by vegetating bacilli within macrophages. Therefore, the proinflammatory status of macrophages, rather than cytolysis, can be considered as a marker of cell susceptibility to LeTx.

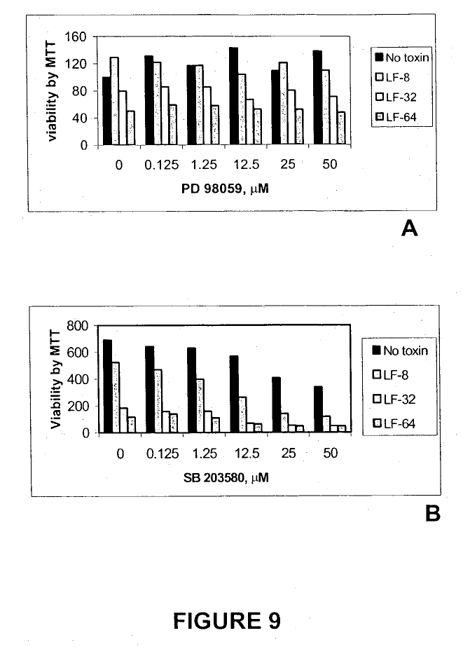

In LeTx-resistant cells, another physiologically relevant LeTx-induced signal or sensitizing mechanism of yet unidentified nature may exist, in addition to the inhibition of an anti-apoptotic survival MAPK pathway by LeTx (Duesbery and Vande Woude, 1998; Vitale et al., 1998; Pellizzari et al., 1999; Pellizzari et al., 2000). Furthermore, data in FIG. 9 shows that the MEK1/2 inhibitor PD98059, and p38 inhibitor SB 203580 in LeTx-treated Raw264.7 cells do not increase cell survival. This is consistent with the model of Duesbery and Vande Woude (1998), wherein MAPKKs cleavage by LeTx leads to the block of downstream activation of MAPK/ERK 1 and 2, rather than to the activation of these kinases, as reported by Vitale et al. (1998).

The invention also provides for inhibitors of LeTx intracellular activity, for example cytokines, inhibitors of caspases, cellular aminopeptidase/5-lipoxygenase, and protein synthesis. These agents decrease the death rate in mice challenged with B. anthracis spores. The pan-caspase inhibitor z-VAD and specific inhibitors of caspases-1 and 3 show similar results.

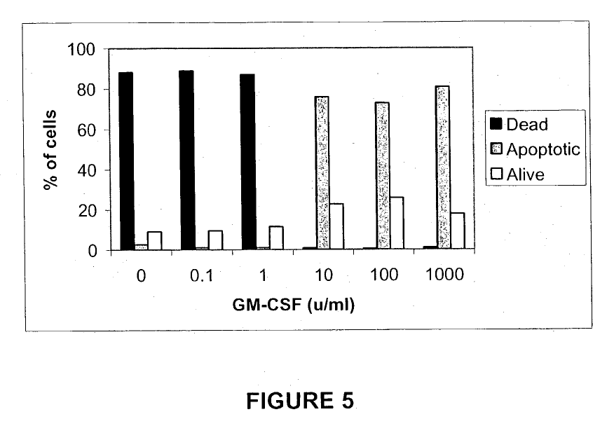

Proteases of the caspase family, especially caspase 1 (ICE), caspase 3, caspase-4 (TX/ICH-2/ICE(rel)II), and caspase-8, are also implicated in Fas (APO-1/CD95)-mediated apoptosis (Kamada et al., 1997). Caspase cleavage of intracellular proteins downstream of caspase-8 activation ultimately results in the disturbance of mitochondrial function and the release of cytochrome c from mitochondria. The release of cytochrome c from mitochondria can induce the activation of an alternative branch of the caspase cascade through the activation of caspase-9. Although both pathways of Fas-mediated caspase activation are functional in most cell types, in some the mitochondrial events are not required for efficient apoptosis (Martin et al., 2002). For example, Hakem et al. (1998) found that Casp9-/-thymocytes were resistant to dexamethasone- and gamma irradiation-induced apoptosis, but were surprisingly sensitive to apoptosis induced by UV irradiation or anti-Fas. Similarly, LeTx-treated cells displayed a strong loss of mitochondrial potential (FIG. 4), though an inhibitor of caspase-9 was unable to protect cells from changes in membrane permeability (FIG. 5).

In another embodiment of the invention, the method of treatment of anthrax infection is inhibition of proteases of the caspase family. This embodiment includes inhibition of caspase 1 (ICE), caspase 3, caspase 4 (TX/ICH-2/ICE(rel)II), caspase 6, and caspase 8. Additionally, inhibitors of caspase-9 can be effective in protecting infected cells from loss of mitochondrial transmembrane potential.

In other embodiments of the invention, general caspase inhibitors can be used. In such embodiments, the caspase inhibitors preferably include, but are not limited to zVAD-fmk and z-YVAD. Other caspases include, but are not limited to, Z-WEHD-FMK for caspase-1, Z-VDVAD-FMK for caspase-2, Z-DEVD-FMK for caspase-3, Z-YVAD for caspase-4, Z-VEID-FMK for caspase-6, Z-IETD-FMK for caspase-8, Z-LEHD-FMK for caspase-9, Z-AEVD-FMK for caspase-10, and Z-LEED-FMK for caspase-13.

Inhibitors of caspases have been described previously (See U.S. Pat. No. 6,355,618). Other caspase inhibitors include M-791, M-920, M-725, and z-VAD. In addition, WO 93/05071 describes peptide ICE (caspase-1) inhibitors. Caspase inhibitors are also described in Popov et al., 2002a, and Bhatnagar et al., 1999.

In yet another embodiment the invention provides for enhancement signaling pathways that lead to protection from stress-induced apoptosis. This embodiment includes enhancement of the ERK (MAPK)-signaling pathway. Furthermore, enhancement of the ERK (MAPK)-signaling pathway by administration of GM-CSF is another aspect of this embodiment.

GM-CSF is a well-known anti-apoptotic cytokine in monocytes (Bingisser et al., 1996; Flad et al., 1999). Preincubation with GM-CSF partially protected Raw 264.7 cells from LeTx (FIG. 5). This finding is consistent with the mechanism of the invention wherein LeTx-induced cell death involves proapoptotic inhibition of cell survival signaling pathways rather than by direct cytotoxic damage causing necrosis (Rathmell et al., 1999). A partial rescue of LeTx-treated cells by GM-CSF could be explained based on findings of Sweeney et al. (1999) that ERK (MAPK)-signaling pathway plays a central role in GM-CSF-mediated protection of neutrophils from stress-induced apoptosis. If the same is true for monocytes, a proapoptotic cleavage of MAPKKs by LeTx should interfere with GM-CSF survival signaling by preventing MAPK activation.

Granulocyte-macrophage colony-stimulating factor (GM-CSF) has previously been extensively studied as a multipotent immunostimulating substance. It displays a number of broad-spectrum beneficial therapeutic properties. A new drug called Leukine® is based on yeast-expressed human recombinant GM-CSF. It was found that Leukine® not only shortens time of white blood cell recovery in cancer patients with allogenic bone marrow transplantation, but also decreases the overall incidence of infection and the length of hospital stays (Immunex Corp. Leukine® Manufacturer Factsheet, 1998). Further studies demonstrated a beneficial effect of GM-CSF administration for a number of bacterial infections. It has low toxicity, can be administered by different routes, and is well tolerated by patients causing a small number of side effects. Recombinant GM-GSF is administered intravenously most preferentially at 6.25 μg/kg/day over a four hour period. Doses up to 100 μg/kg/day can be administered.

Serum withdrawal in the presence of IFN-γ substantially increased PBMC's susceptibility to LeTx (FIG. 2 ). Conditions of serum starvation are known to increase Fas-mediated apoptosis in human diploid fibroblasts (Ahn et al., 2001), and to activate stress-responsive JNK pathway in neuronal cells (Le-Niculescu et al., 1999). Fas activated p38 and JNK pathways are also present in Jurkat cells (Juo et al., 1997). The latter pathway is known to be required for induction of FasL promoter activity in response to various stress stimuli (Faris et al., 1998). Our data on MEK1/2 inhibitor PD98059, and p38 inhibitor SB 203580 in LeTx-treated Raw264.7 cells show that none of these inhibitors increased cell survival (FIG. 9). This is consistent with the model of Duesbery and Vande Woude (1998), wherein MAPKKs cleavage by LeTx leads to the block of downstream activation of MAPK/ERK 1 and 2, rather than to the activation of these kinases, as reported by Vitale et al. (1998). In LeTx-resistant cells, another physiologically relevant LeTx-induced signal or sensitizing mechanism of yet unidentified nature may exist, in addition to the inhibition of an anti-apoptotic survival MAPK pathway by LeTx (Duesbery and Vande Woude, 1998; Vitale et al., 1998; Pellizzari et al., 1999; Pellizzari et al., 2000).

In one embodiment the invention provides for a method of treatment for anthrax infection by inhibiting toxin intracellular activity. In a further embodiment, the invention presents a method of treatment for anthrax infection by block the entry of LeTx into susceptible cells.

In addition, the invention provides an antimicrobial treatment by inhibiting toxin intracellular activity through enhancement of the activity of infected macrophages. This aspect of the invention allows for a decrease in intracellular bacterial survival. In general, inhibition of LeTx activity within macrophages is an early treatment of anthrax preventing the initiation of infection.

In another aspect of the invention, a method of treating inhibiting LeTx activity in cells is provided by treating the cells with at least one macrophage. deactivating cytokine. These cytokines include: IL-4, IL-6, IL-10, TGF-β1, MIP-α, MIP-1β, and RANTES, and mixtures thereof.

The invention also encompasses cytoplasmic delivery vehicles, such as liposomes and microcapsules. Studies on LeTx-induced pathogenesis in infected macrophages also require a model of intracellular toxin expression. pH-sensitive liposomes (Lutwyche et al., 1998; Cordeiro et al., 2000) can be used for the delivery of LeTx into phagocytes.

Different formulations of liposomes are available (Drummond et al., 2000). In particular, pH-sensitive phosphatidylethanolamine/cholesterylhemisuccinate liposomes (Lee et al., 1996; Cordeiro et al., 2000) are useful. The amount of protein encapsulated is monitored by flow cytometry of liposomes with fluorescently labeled PA. The same liposomes are used to estimate the efficacy of cytoplasmic delivery of toxin by measuring the intensity of cellular fluorescence by flow cytometry in cell fusion experiments. Apoptosis and lysis of cells is evaluated by standard techniques. Dose-response curves are obtained, and relative cell sensitivity to toxin is compared with results obtained using an extracellular protocol. The liposome delivery is a more adequate representation of intracellular toxin expression independent of toxin-receptor interactions. Cells previously thought to be insensitive to toxin can show increased LeTx susceptibility when delivered by liposomes. These effects have only been considered in one early paper by Singh et al. (1989) on pinocytotic delivery.

Furthermore, liposomes can be used for targeting cellular signaling pathways in B. anthracis (Sterne) infected macrophages. To demonstrate this, cells are infected at a MOI of 20 for 30 min. The cells are then washed and extracellular bacteria inactivated with gentamicin at 30 mg/ml for 1 h. At different time points up to 5-6 h, cells are treated with bafilomycin A1. At the end, cells are washed, lysed with saponin, and the viable bacterial content is estimated by plating onto agar.

The invention also includes the development of intracellular prophylactic and therapeutic treatments for anthrax consisting of a cocktail of antibiotics and inhibitors. J774A.1 or similar cells are treated with LeTx at concentrations ranging from 1-50 ng/ml for 1 to 4 h. The cells are washed and treated with liposome formulations determined from assays of lethal toxin inhibition. For example, 10-100 μM bafilomycin A1, an inhibitor of vacuolar proton ATPases (Bowman et al., 1988). In the presence of bafilomycin A1, phagosomes to maintain a steady alkaline pH and prevent vacuolar perforation by pore forming toxins (Porte et al., 1999; Rathaman et al., 1996) can be used. Acidification of macrophage phagosomes by treatment with LeTx labeled with pH-sensitive dyes, such as NHS-rhodamine or Oregon Green 488-rhodamine (Molecular Probes) can also be used. The change in intracellular pH after phagocytosis is monitored by fluorescent microscopy or flow cytometry both before and after bafilomycin treatment.

A spectrum of inhibitors are known to inhibit phagosome function and are widely used in anti-microbial and anti-parasitic therapies, such as the lysosomotropic amine chloroquine and its derivatives, the carboxylic ionophore monensin, and newer drugs (Weber et al., 2000; Iacoangeli et al., 2000; Drose et al., 2001). These can be used for anti-LeTx therapy, but they may have proapoptotic properties in certain cell types (Hashimoto et al., 2001). Use of these agents is preferably avoided.

In yet another embodiment of the invention, inhibitors of matrix metalloproteases (MMPs) are provided as inhibitors of the surface shedding of transmembrane FasL, which can inhibit LeTx activity. Matrix metalloproteases are involved in FasL processing from transmembrane to apoptosis-potentiating soluble form (Powell et al., 1999; Hidalgo and Eckhardt, 2001). LeTx is structurally similar to MMP, and the biological relevance of its substrates (MAPKKs) to apoptosis has not yet been directly demonstrated (Pellizzari et al., 1999). The role of sFasL in the induction of apoptosis, compared to membrane-bound FasL, is controversial and probably depends on cell type and other factors (Powell et al., 1999; Suda et al., 1997). A role of shedding in LeTx activity, provides a mechanism of inhibition of apoptotic death.

Use of broad-spectrum MMPIs, BB-94 (batimastat) and BB-2516 (marimastat), both of which are currently under clinical trials, and newer ones, BB3103 and A-151011 (all from British Biotech, UK), are provided in this embodiment of the invention. Toxin-sensitive monocyte/macrophages are transferred to medium with 0.05% fetal calf serum and inhibitor is added for 24-48 h at a concentration of 0.1-1 mM (BB-94 and BB-2516) or 5-50 μM (BB-3103 and A-151011). The relative amount of surface FasL expression is determined by flow cytometry by surface staining the cells with FITC-conjugated specific anti-FasL antibody. As a positive control, a Fas-neutralizing antibody able to crosslink Fas molecules and increase apoptosis, such as anti-human CH11, ZB4 (Panvera), or anti-mouse TNFSF6 (R&D Systems) antibodies, are used at a concentration of about 5 μg/ml. Control experiments will employ an isotype-matched control.

Examples of MMPIs include BAY 12-9566 (Rowinsky et al. 2000); marimastat (Groves et al. 2002); COL-3 (Cianfrocca et al. 2002); and carboxylate ester compounds (see WO 92/09563, U.S. Pat. No. 5,183,900, U.S. Pat. No. 5,270,326, EP-A-0489577, EP-A-0489579, WO 93/09097, WO 93/24449, WO 94/25434, WO 94/25435, WO 95/04033, WO 95/19965, and WO 95/22966). BAY 12-9566 can be administered at daily oral doses ranging from 100 to 1,600 mg, once to four times daily. A typical dosing regime of marimastat is 50 mg per day during a 28-day cycle. COL-3 can typically be administered orally once daily.

LeTx itself can also be inhibited by one of the known MMPIs. Relevant controls for LeTx inhibition in a MEK2 cleavage assay as described by Pellizzari et al. (1999) demonstrate this characteristic of the invention. Briefly, RAW264.7 cells are treated with LeTx in presence or absence of inhibitor, lysed, and the lysate is tested by Western blotting using an MEK-specific antibody, such as N-20 (Santa Cruz Biotechnology).

In an embodiment of the invention, GM-CSF is administered as an adjunct to low doses of antibiotic therapy, including, but not limited to, neomycin.

In one embodiment, bestatin (Ubenimex, NK421), or a pharmaceutical composition comprising Ubenimex, can be used as a treatment for anthrax. Ubenimex is administered by various routes, although oral administration is preferred. The dosage for administration, depends on the age, sex, and weight of patient, degree of infection, and administration route etc. A typical dose is 10-100 mg per day.

In another embodiment, other inhibitors can be used to provide actions similar to that of bestatin. One example is the dihydroxy fatty acid leukotriene B₄ (LTB₄). LTB₄ is produced by the leukotriene cascade of arachadonic acid, which is a key mechanism in many inflammatory and allergic disease states. LTB₄ stimulates adhesion of circulating neutrophils to vascular endothelium, directs their migration toward sites of inflammation, and induces secretion of further inflammatory mediators. In addition, Leukotriene-A₄ hydrolase (LTA₄-hydrolase) (EC 3.3.2.6) is an enzyme that catalyzes the final and rate limiting step in the synthesis of LTB₄. Inhibition of LTA₄ hydrolase selectively blocks the biosynthesis of LTB₄, which may provide an advantage over current inhibitors, such as those of 5-lipoxygenase, that block earlier in the leukotriene cascade and as a result are less selective. PCT/GB99/00284 provides a method for treating mammals by inhibiting leukotriene-A4 hydrolase activity, comprising administering to the mammal an amount of a compound of general formula or a pharmaceutically acceptable salt hydrate, or solvate thereof, sufficient to inhibit such activity.

In another embodiment of the invention, these treatments of the previous embodiments can be combined with traditional antibiotics for a combined therapy against anthrax infection.

In yet other embodiments of the invention, these and other inhibitors of lethal toxin intracellular signaling can be used in combination with each other and with antibiotics. In such embodiments, the antibiotics include, but are not limited to, ciprofloxacin.

Inhibitors of lethal toxin intracellular signaling of the invention preferably are z-VAD and z-VAD, though other methylated derivatives of bestatin with higher bioavailability are also useful.

The antibiotic ciprofloxacin is administered to patients either orally or intravenously. Oral administration can be at a dose of from 100 mg to 750 mg, including 250 mg, and 500 mg, every twelve hours. In children, oral administration of ciprofloxacin is 15 mg/kg per dose up to 500 mg per dose. Intravenous ciprofloxacin is administered every twelve hours in doses ranging from 200 to 400 mg. In children ciprofloxacin is administered intravenously at 10 mg/kg up to 400 mg per dose. Treatment with ciprofloxacin lasts from 5 to 60 days. Neomycin can be administered at doses from 1 to 10 mg/kg/day.

In embodiments of the invention, zVAD can be administered at doses from 1 to 45 mg/kg/day, 20-30 mg/kg/day, 15-25 mg/kg/day, or up to 50 mg/kg/day. Bestatin can be administered at doses ranging from 1 to 45 mg/kg/ day, 20-30 mg/kg/day, 15-25 mg/kg/day, or up to 50 mg/kg/day.

Lethal toxin-induced apoptosis in macrophages and human blood cells. Substances, previously known as protecting cells from lethal toxin action, such as bestatin and neomycin, act as inhibitors of apoptosis. In addition, caspase inhibitors protect cells from toxin-induced apoptotic death. However, the effect of lethal toxin inhibitors, administered alone, was not enough to confer full protection from the infection, therefore bestatin was not previously considered as an anthrax treatment. The experiments on which the invention is based demonstrate that the effect of toxin-signaling inhibition is synergistic with the general antibacterial effect of antibiotics, such as ciprofloxacin. As a result, a combined therapy can provide full protection against the disease at lower doses of antibiotics and allows a broader window of opportunity for the successful administration of antibiotics.

Therefore, in an embodiment of the invention, the combined treatments can be used to treat antibiotic-resistant anthrax infection.

Microbe-induced apoptosis is quite common in infections of different origin. For example staphylococcal infections, yersinioses of different etiology, hemorrhagic fevers, exhibit apoptosis. Embodiments of the current invention utilizes a combined therapy of anti-apoptotic and anti-microbial treatments for anthrax infection.

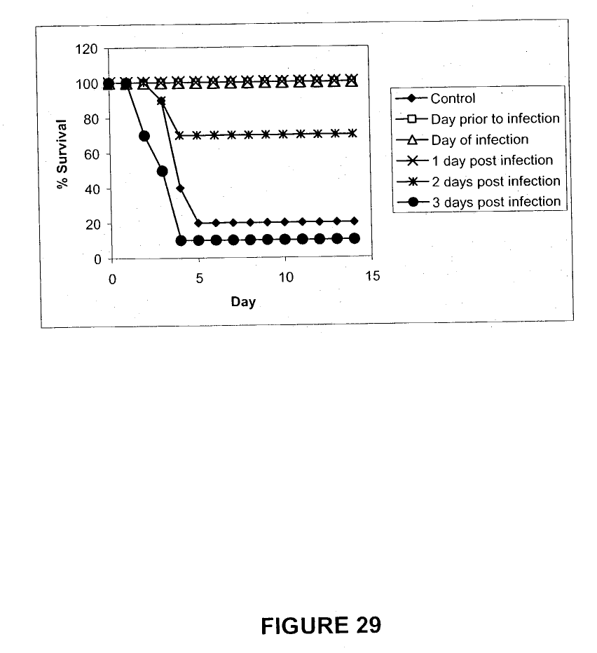

The treatments of an embodiment of the invention provide a combination of lethal toxin inhibitors with antibiotics, and therefore target both the infected cells of the patient that have been exposed to toxins and the microbial cells. The synergism between the two components of the invention allows relatively low doses of the toxin inhibitors to be used for treatment and prophylaxis, and also allows for possible delays in the use of antibiotics. Delayed use of antibiotics is especially important because delays in confirmation of exposure due to a biological attack are often expected. Thus, antibiotic treatment alone may be ineffective. To illustrate this disadvantage of antibiotic treatment alone, FIG. 29 demonstrates the survival of mice after a delayed treatment with ciprofloxacin. Whereas the treatment on the day of infection resulted in a complete protection, a delay in 2 days resulted in only 10% survival.

Embodiments of the current invention provides a combined treatment which can be used in different situations. In one embodiment the treatments of the invention can be used prophylactically. In another embodiment, antibiotics are administered either immediately after exposure to the infectious agent (immediate post-exposure treatment) or after a certain delay (late identification followed by late treatment). In all cases, the anti-toxin therapy is carried out simultaneously with antibiotics. Generally, anti-toxin treatment is administered from the beginning of the infectious process.

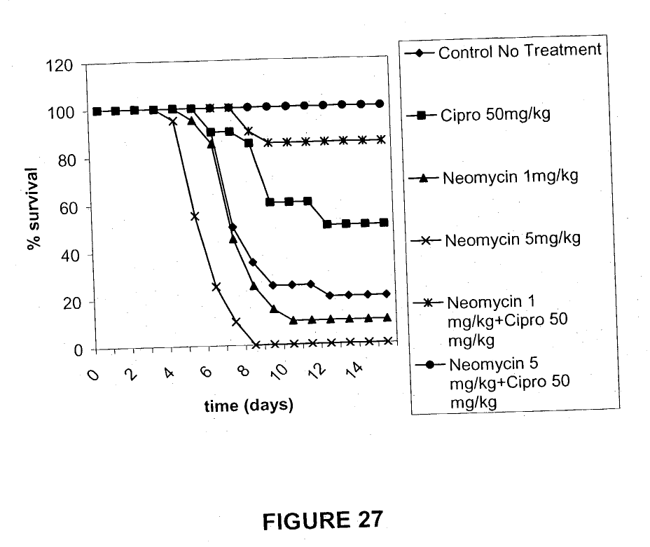

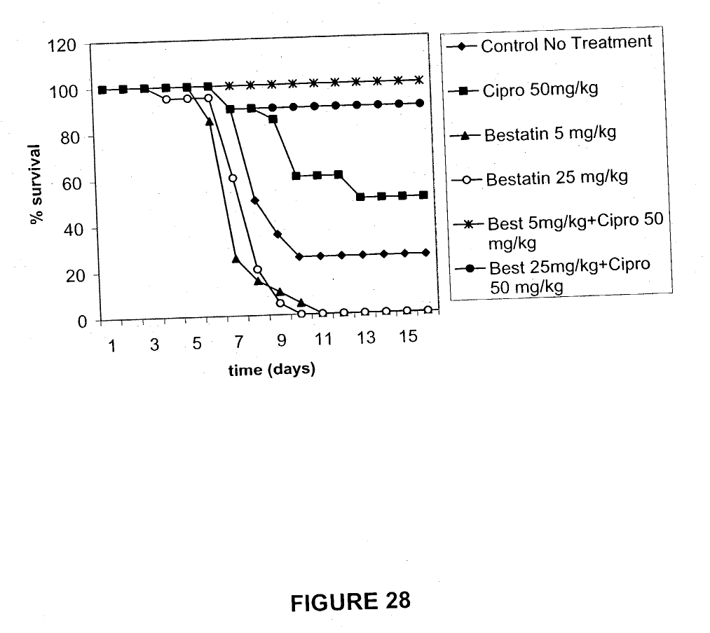

When antibiotic had been delivered late after the exposure, typically when treatment is with antibiotic alone, the prognosis is poor. In contrast, when antitoxin pretreatment is combined with late antibiotic administration, full protection is provided (FIGS. 27 and 28). To demonstrate this, mice were treated with the indicated amounts of inhibitors starting day −1 and continued through day +10. At the day 0 they were challenged with the indicated amounts of anthrax (Sterne) spores. Antibiotic treatment (ciprofloxacin) started at day 2, and continued though day +10. Mortality was recorded until day 15. These data indicate that in the case when a biological weapon attack is suspected, or the exposure may have already occurred, but has not been confirmed, a low-dose administration of a prophylactic anti-toxin medication is indicated. After exposure has been confirmed, the delayed (but still effective) antibiotic therapy is initiated. Currently, late antibiotic administration alone is ineffective.

In addition, these embodiments of the invention provide for lower doses of antibiotic in combined therapy, thus avoiding the harmful side effects of these antibiotics.

The term “antibiotic-resistant anthrax infection” refers to a strain of anthrax against which antibiotic therapy does not provide complete protection in conditions where the equal therapy provides complete protection against another strain. This latter strain is termed “antibiotic-sensitive anthrax.”

The term “low-dose” refers to a dosage of antibiotic that is below the therapeutically effective amount.

In another embodiment, the invention provides a mechanism for inducing the proinflammatory response, which is inhibited by LeTx. This embodiment includes the administration of IL-1β, TNF-α, and IL-6 to compensate the decreased production in the presence of LeTx.

In summary, the invention is based on a novel mechanism of LeTx-induced cell death in mouse and human cells. Elucidation of the bacterial molecules and the mechanisms by which B. anthracis trigger apoptosis of host cells provides valuable information for new approaches to disease treatment.

These examples demonstrate the cytopathological effects of LeTx in human PBMCs and its relevance to human disease, which are the focus of this invention. The data on which the invention is based provide that anthrax infection in both mouse (Popov et al. 2002a) and human phagocytes leads to the reduction in their bactericidal capacity against spores and germinating bacilli, and the appearance of apoptotic cells. Apoptotic changes can also be found in LeTx-treated human PBMCs and monocytes.

The examples describing this invention are aimed at the cytopathological effect of LeTx in a range of concentrations (including nonlytic) in two different systems. The first system is a mouse cell line, Raw 264.7, and is the most susceptible to LeTx and the best studied. The second system is human PBMCs, which are resistant to LeTx, and the most relevant to human disease.

This invention will be described in greater detail in the following Examples.

### Materials and Methods

The Examples demonstrating this invention were conducted using the following materials and methods:

Materials. Alamar Blue® (Biosource, USA), bestatin, dimethyl sulfoxide, MTT (3-(4,5-Dimethyl-2-thiazolyl)-2,5-diphenyl-2H tetrazolium bromide), and saponin from Quillaja bark (Sigma/Aldrich, USA), Cellstripper® (Mediatech, USA), Fico/Lite-LymphoH (Atlanta Biologicals, USA), DMEM/F12 media, AIM-V® serum free media, penicillin-streptomycin, phosphate buffered saline solution (Gibco, USA), YO-PRO®-1/propidium iodide Stain: Vybrant® Apoptosis Assay Kit #4, JC-1 and JC-9 mitochondrial potential sensors (Molecular Probes, USA), In Situ Cell Death Detection Kit (Roche Molecular Biochemicals, USA), staurosporine (Alexis Biochemicals, USA), PD98059 and SB203580 (Calbiochem, USA) (Sigma, USA), Caspase Inhibitor Sampler Pack, recombinant human INF-γ, recombinant mouse INF-γ, recombinant mouse GM-CSF, anti-mouse Fas Ligand/TNFSF6 antibody, anti-mouse FAS fluorescein-labeled antibody (R&D Systems, USA).

Lethal Factor and Protective Antigen LF and PA were isolated as described elsewhere (Leppla et al. 1988; Park, et al. 2000) and were stored at −70° C. in 10 μg/ml stock solutions. B. anthracis (Sterne) spores were prepared in LB agar broth. After a ratio of spore to vegetative bacteria reached 99:1, the spores were pelleted, washed five times with distilled water, and the concentration was adjusted to 1×10⁹ spores/ml.

Flow cytometry. Experiments were carried out in a FACSCalibur™ Becton Dickinson Immunocytometry System.

Cells and cell culture. Mouse macrophages, RAW 264.7, were obtained from the American Type Culture Collection, (ATCC TIB-71, Manassas, Va.). RAW 264.7 cells were cultured in DMEM/F12 medium with phenol red, which was supplemented with 2 mM glutamine, 10% heat-inactivated fetal bovine serum, 10,000 units/mL streptomycin-penicillin, 0.1 mM of non-essential amino acids, and 0.5 mM 2-mercaptoethanol. The cells were maintained in an incubator at 37° C. in a 5% CO₂ atmosphere. Phosphate buffered saline, pH 7.4, was used to wash the cells, and Cellstripper® was used to remove the cells from the flask surface according to manufacturer's instructions. RAW 264.7 cells were not allowed to go through more than 90 passages.

Human peripheral blood mononuclear cells (PBMCs) and monocytes were isolated from the whole blood obtained from Red Cross volunteers (Rockville, Md.) were isolated using Fico/Lite LymphoH (Coligan et al., eds, 1999). Monocytes were further isolated from PBMCs by cell attachment onto a tissue culture dish for 1 h. The supernatant was washed away, and the adherent fraction containing isolated monocytes was removed using Cellstripper. PBMCs and human monocytes were cultivated in DMEM/F12 without Phenol Red and penicillin, and were maintained in an incubator at 37° C. in 5% CO₂.

Cell treatment with toxin. Murine cells were treated with LF at varied concentrations from 4 to 64 ng/ml while human cells received 50 to 500 ng/ml. PA concentration was keep constant at 500 ng/ml. Toxin was prepared immediately before the experiment. The toxin was never refrozen or used after 24 h after being defrosted. Human cells were preactivated with a recombinant INF-γ (100 U/ml) for at least 24 to 48 h.

Apoptosis assay. For staining with YO-PRO®-1/propidium iodide the cells were stripped from plastic and suspended in 900 μl of PBS. Then, 100 μl of stain was added. Staining was carried out at 4° C. for 30 min. Stain was prepared according to manufacturer's instructions. For the JC-1 staining, cells were stripped and suspended in 1 ml of working solution of 10 μg/ml in PBS. Staining was carried out at 4° C. for 10 minutes. At least 4000 cells were counted for each point.

Staurosporine was used as an inducer of apoptosis in human and mouse systems at 50 μM and 5 μM, respectively.

Tunel assay. The assay was used to measure DNA fragmentation during apoptosis. The technique uses deoxynucleotidyl transferase to incorporate labeled nucleotides to apoptotic DNA strand breaks in situ. Cells were striped from plastic and suspended in 100 μl of PBS. The samples were prepared for Tunel according to manufacturer's instructions (Roche Molecular Biochemicals, USA).

MTT Assay. MTT was used to analyze cell viability of RAW 264.7 macrophages after treatment with LeTx. MTT was diluted in phosphate buffered saline and then added to cells at 1 mg/ml. After 1 h of incubation the supernatant was removed and developer was added. MTT developer consisted of 91% (v/v) isopropanol, 4% (v/v) of 1M HCl, and 5% (v/v) of 10% (w/v) sodium dodecyl sulfate. Spectrophotometric readings were then taken using μ-Quant (Bio-Tek Instruments, Inc., USA).

Caspase inhibitors. The following inhibitors were used: Z-WEHD-FMK for caspase-1, Z-VDVAD-FMK for caspase-2, Z-DEVD-FMK for caspase-3, Z-YVAD for caspase-4, Z-VEID-FMK for caspase-6, Z-IETD-FMK for caspase-8, Z-LEHD-FMK for caspase-9, Z-AEVD-FMK for caspase-10, and Z-LEED-FMK for caspase-13. The inhibitors were diluted in DMSO according to manufacturer's instructions. RAW 264.7 cells (1×10⁶ cells/well) were preincubated for 15 h in media with 20 μM inhibitor. Then, LeTx (4 ng/ml of LF, 500 ng/ml of PA) was added to the culture. After 4 h of toxin treatment, cells were stripped and stained YO-PRO®-1/propidium iodide stain.

Blocking of Fas-L with antibody. RAW 264.7 (1×10⁶ cells/well) received 1 ml of media with anti FasL neutralizing antibody. Immediately, LeTx (4 ng/ml of LF, 500 ng/ml of PA) was added to the culture. After, 15 h of toxin treatment cells were stripped and stained YO-PRO®-1/propidium iodide stain.

Phagocytic and bactericidal activity of LeTx-treated cells. For measuring spore phagocytosis by macrophages, RAW 264.7 were grown in media without phenol red or antibiotics (2×10⁵/well), then spores were added and phagocytosis was allowed for 30 min. In preliminary experiments the phagocytosis time of 30 min was found to be approximately a half-time of maximal phagocytosis providing the maximum sensitivity and linearity of the assay before cells engaged actively in bacterial killing. Then the supernatant was removed, and cells were washed with equal volume of phosphate buffered saline six times. It has been shown that this washing procedure removes more than 85% of spores from control wells. Finally, the cells were lysed with 1% aqueous saponin for 5 min. After lysis Alamar blue® in media without Phenol Red was added to each well, and the fluorescence readouts were taken according to manufacturer's instructions. In control experiments, fluorescence intensity was shown to be linear proportional to the concentration of spores in the range 1×10⁴ to 1×10⁶ spores/well.

Measurement of Killing Activity. For measuring killing activity of RAW 264.7 macrophages and human PBMCs the procedure was similar to that described above, except that the cells were first treated with bestatin for 1 h, and incubated with spores for 3 h. The plate was spun, supernatant removed, and Alamar Blue® readouts taken. Human PBMCs (2×10⁵/well) were prestimulated with recombinant human INF-γ (100 u/ml) for 24 h in the absence of antibiotics before the addition of bestatin. Distilled water was used for cell lysis for 20 min.

Statistical analysis. Student's t-test were performed with error bars corresponding to +/−95% confidence.

PBMCs and Monocytes PBMCs and monocytes were isolated from whole blood obtained from American Red Cross volunteers (Rockville, Md.) using Fico/Lite LymphoH (Atlanta Biologicals, USA), according to established procedures (Coligan et al. 1999). Monocytes were further isolated from PBMCs by cell attachment onto a plastic tissue culture dish for 1 h. The supernatant was washed away, and the adherent fraction containing isolated monocytes was removed using a CellStripper® solution. Alternatively, monocytes were negatively selected using a magnetically activated cell sorting (MACS) cell isolator (Milteny Biotec Inc., Germany). PBMCs and monocytes were cultured in DMEM/F12 supplemented with 10% fetal bovine serum, 2 mM L-glutamine, 100 U/ml penicillin, 100 μg/ml streptomycin (Sigma-Aldrich, USA) at 37° C. in 5% CO₂. Phenol Red and antibiotics were omitted when indicated. Human cells were treated with 50 to 500 ng/ml of LF. The PA concentration was kept constant at 100 or 500 ng/ml. When indicated, cells were preactivated with recombinant INF-γ (100 U/ml; R&D Systems, USA) for at least 24 to 48 h. After incubation at 37° C. (5% CO₂) for 48 h, the supernatants were collected and stored at −80° C.

YO-PRO®-1/propidium iodide Staining For staining with YO-PRO®-1/propidium iodide (Vibrant Apoptosis Assay Kit #4, Molecular Probes, USA), cells were stripped from the plastic tissue culture dish and suspended in 900 μl of PBS with 100 μl of stain (prepared according to manufacturer's instruction). Staining was carried out at 4° C. for 30 min. At least 4000 cells were counted for each sample over a constant period of time. For JC-1 staining, cells were stripped and suspended in 1 ml of working solution of 10 μg/ml in PBS. Staining was carried out at 4° C. for 10 minutes. Staurosporine (Alexis Biochemicals, USA) was used as an inducer of apoptosis.

### Example 1

### Sublytic Concentrations of LeTx Cause Apoptosis-like Changes in Cellular Cytoplasmic and Mitochondrial Membranes

The mouse macrophage cell line RAW 264.7 is sensitive to LeTx, and is widely used in anthrax studies (Hanna et al., 1993). At concentrations close to 100 ng/ml of LF in presence of PA (usually 100 to 500 ng/ml), RAW 264.7 cells undergo quick lytic death (Hanna et al., 1993). However, much lower concentration of the toxin were reported to inhibit cytokine production in cells (Erwin et al., 2000, Hanna et al., 1993, Shin et al., 2000). This Example demonstrates how LeTx, at sublytic concentrations, may interfere with cell signaling.

In order to detect changes in plasma membrane permeability, monolayers of RAW 264.7 cells were treated with a range of LeTx concentrations, and screened for apoptotic changes by flow cytometry using a green fluorescent dye (YO-PRO-1, Molecular Probes) capable of detecting early apoptosis-specific changes in membrane permeability and composition, and a late apoptosis/necrosis-specific red fluorescent dye (propidium iodide, PI).

Incubation of cells with PA alone (500 ng/ml) or in combination with sublytic concentrations of LF causes a shift in green fluorescence intensity, characteristic of apoptosis (FIG. 1). At an LF concentration of 4 ng/ml a shift in green fluorescence indicative of early apoptotic changes was observed. As the concentration of LF was increased to 8 ng/ml, both green and red fluorescence greatly increased without substantial lysis, indicating the onset of late apoptotic events. Lytic effects were observed at LF concentrations of 32 ng/ml and higher, as indicated by a shift towards lower green fluorescence and an overall lower cell count (data not shown), usually associated with a disintegration of cells into the apoptotic bodies (Wilson et al., 1999).

Studies of the effect of LeTx on human monocyte/macrophage cells isolated from peripheral blood provide a better understanding of human cell behavior after anthrax infection. Human cells showed a considerably higher resistance to LeTx compared to RAW 264.7 cells. At the highest concentration of LeTx tested (500 ng/ml LF and 500 ng/ml PA), no lysis was detected in human monocyte/macrophage cells under the same media conditions used for RAW 264.7 cells (FIG. 2A). After 48 h stimulation with IFN-γ, a certain number of dead cells were detected, as indicated by the presence of a population of cells with high both red and green fluorescences (the second peak in FIG. 2A). Cells treated with staurosporine as a positive control (Roucou et al., 2001) showed a fluorescence pattern typical of apoptotic cells (FIG. 2A). However, human monocytes became considerably more susceptible to LeTx in the serum starvation conditions (FIG. 2B). The amount of cells acquired green fluorescence relative to untreated control increased, while the amount of double-stained cells decreased, indicating a change in cell death mode.

DNA fragmentation is a typical feature of apoptotic death (Cohen, et al., 1994). The Tunel assay, which is based on the incorporation of fluorescent substrate into the ends of DNA fragments with the aid of terminal deoxyribonucleotidyl transferase confirmed that apoptotic death takes place in cells in the presence of LeTx. Increased fluorescence of LeTx-treated cells was detected compared to untreated control, in Tunel assay (FIG. 3).

Mitochondria are known mediators of the ‘intrinsic’ apoptotic pathways (Rathmell et al., 1999). Studies of mitochondrial involvement in LeTx-induced cell death using a specific dye, JC-1, capable of changing color depending on mitochondrial transmembrane potential demonstrate this role. RAW 264.7 cells were treated with LeTx and subsequently stained with JC-1. The transmembrane potential of live mitochondria decreased upon LeTx treatment, consistent with the onset of apoptosis (FIG. 4). Staurosporine, a drug known to cause mitochondrial toxicity (Roucou et al., 2001), caused JC-1 staining changes similar to LeTx.

### Example 2

### GM-CSF Modulates Cell Survival in Presence of LeTx

The ability of cytokines to modulate apoptosis suggests that some cytokines may differentially influence the outcome of LeTx activity in cell. This was tested in studies on the susceptibility of RAW 264.7 cell to LeTx in the presence of murine granulocyte-macrophage colony stimulating factor (mGM-CSF), a cytokine known to confer partial protection against apoptosis (Flad et al., 1999; Sweeney et al., 1999). Incubation of cells with LeTx (32 μg/ml) led to lysis of the majority of cells, as reflected by a low green fluorescence channel count (FIG. 5). In presence of mGM-CSF, overall cell survival, as well as the relative number of cells undergoing apoptosis versus the combined numbers of dead and alive cells, was substantially increased. This suggests an inhibitory effect of GM-CSF on LeTx-induced apoptosis and cell lysis. GM-CSF, however, was incapable of preventing apoptosis completely. Instead, the progression of cellular events from apoptosis to lysis was retarded, leading to the increase in the relative amount of apoptotic cells.

### Example 3

### Inhibition of Caspases Decreases LeTx Pathogenicity

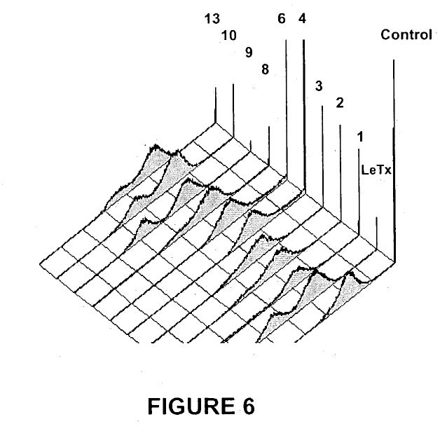

Apoptosis is carried out via the action of a number of initiator and effector caspases (Rathmell et al., 1999). Inhibition of caspases using specific inhibitors of caspases 1, 2, 3, 4, 6 and 8 leads to different degree of protection of cells against LeTx, with inhibition of caspases 4, 6 and 8 being the most active (FIG. 6). This finding is consistent with a model of LeTx-assisted apoptosis initiated at the level of death receptors, and often involving caspase 8 activation. The latter is usually followed by the activation effector caspases, such as caspases 3 and 6 (Rathmell et al., 1999). Caspase 9 was not activated in spite of established LeTx-induced mitochondrial damage (FIG. 4).

### Example 4

### Fas/Fas Ligand Interaction is Involved in Lethal Toxin Activity

The studies of caspase inhibition in LeTx-treated cells (Example 3) suggest that cellular death receptors are involved. To decipher the LeTx signaling cascade, it is important to determine which of the major apoptotic pathways (TNF-α, FasL, TRAIL/Apo2L, or Apo3L-induced) (Walczak and Krammer, 2000) is targeted by LeTx. The TNF-α pathway can be ruled out based on published data showing a decrease in LPS-stimulated TNF-α production in the presence of LeTx (Pellizzari et al., 1999). TRAIL/APO-2L is a newly identified member of the TNF family that induces apoptosis in cancer cells without affecting most non-neoplastic cells, both in vitro and in vivo (Walczak and Krammer, 2000). The Fas/FasL system is the best characterized of the apoptosis (Ashkenazi et al., 2001). LeTx-induced Fas-mediated apoptosis is explored in this Example. Extracellular staining for Fas using fluorescein-labeled specific antibodies showed that RAW 264.7 cells express detectable amount of Fas which were greatly increased after stimulation with mIFN-γ, mTNF-α, and mGM-CSF (FIG. 7). However, treatment of the cells with LeTx did not induce Fas expression (data not shown).

In order to show that Fas/FasL interaction is involved in the LeTx induced cell death, specific anti-FasL antibodies (TNFSF6) raised against the extracellular domain of recombinant murine FasL were used. When added to cells this antibody is capable of neutralizing activity of recombinant sFasL. LeTx cytotoxicity in RAW 264.7 cells was effectively abrogated by the anti-FasL antibody treatment (FIG. 8). In the absence of the antibody, cells were almost completely killed by an overnight incubation with LeTx. Incubation with the antibody greatly reduced the amount of dead cells, whereas the amount of apoptotic cells increased indicating a substantial delay in the execution of apoptosis. Higher antibody concentrations showed, however, some increase in cell death possibly due to crosslinking by the FasL.

### Example 5

### MAPKK Inhibitors do not Interfere With LeTx Signaling

The apoptotic processes in macrophages can be downregulated by MAP kinases of the ERK subgroup to extend the lifespan of cells (Tudan et al., 2000). It has been shown that a dynamic balance between MAPKK activated ERK and stress-activated JNK-p38 pathways may be important in determining whether cells survive or undergo apoptosis (Xia et al., 1995). LeTx is a metalloproteinase capable of cleavage of MAPKK enzymes (Duesbery and Vande Woude, 1999; Pellizzari et al., 1999; Vitale et al., 1998), but it remains uncertain whether this cleavage leads to inactivation (Pellizzari et al., 1999) or stimulation (Duesbery and Vande Woude, 1998) of MAPKs. If inhibition of MAPKKs by LeTx takes place, the presence of MAPKKs inhibitors would not be expected to decrease the cytotoxic effect of LeTx. Indeed, a specific MAPKK1/2 inhibitor, PD 98059, did not display an antitoxin effect (FIG. 9A). Another MAPK, p38, often provides cell survival signaling along a different pathway (Yamaguchi et al., 2001). A p38 inhibitor, SB 203580, was not able to decrease the effect of LeTx on RAW 264.7 cells at a wide range of concentrations (FIG. 9B). These data are consistent with the occurrence of LeTx-induced apoptosis via inactivation of an anti-apoptotic MAPKKs 1/2, and also indicate that the p38 pathway does not provide a proapoptotic signaling in RAW 264.7 cells in presence of LeTx.

### Example 6

### Inhibition of LeTx Increases Bactericidal Activity of Human PBMCs

Elucidation of the proapoptotic function of LeTx described in the previous Examples, combined with the observation that the LF, EF and PA genes are expressed early in the infection process (Guidi-Rontani et al., 1999), provide the basis for a demonstration that apoptotic changes in spore-infected RAW 264.7 cells. In cells stained with apoptotic dyes at different time points after the addition of spores, gating of FACS scatter plots allows almost complete separation of signals from spores/vegetative bacilli, and infected cells on fluorescence plots (FIG. 10). Within 5 h of infection the cell population corresponding to uninfected macrophages disappeared (FIG. 10E-F), while the intensity of cell stained positively for apoptosis-like membrane permeability considerably increased. Compared to the uninfected cells (FIG. 10C), a scatter plot of the infected cells (FIG. 10D) considerably changed indicating the appearance of cells with a smaller size and a more irregular surface, as it could be expected from the apoptotic cell morphology. The apoptotic changes in macrophages explain the ability of LeTx to reduce production of proinflammatory cytokines by stimulated cells, and implicate LeTx as an early intracellular virulence factor secreted by vegetating bacilli within macrophages. LeTx could therefore cause a reduction in phagocytic and/or bactericidal activity of macrophages against spores and germinating bacilli. RAW 264.7 cells treated with sublytic concentration of LeTx have decreased phagocytosis of B. anthracis (Sterne) spores (FIG. 11). In addition, LeTx function within infected macrophages could be relevant to their reduced antimicrobial activity. Survival of anthrax spores in infected human PBMCs and RAW 264.7 cells treated with different amounts of bestatin, a known inhibitor of intracellular signaling induced by LeTx (Menard et al., 1996) was demonstrated. In the presence of bestatin, the killing activity of PBMCs increased in a concentration dependent manner (FIG. 12). Under the conditions of the experiment the intrinsic antibiotic activity of bestatin was undetectable (data not shown).

### Example 7

### IL-4 Treatment Increases Cell Viability in RAW 264.7 Cells Treated With LeTx

To demonstrate the differential effects of cytokines on the cell viability of RAW 264.7 cells exposed to LeTx, viability after treatment with twelve different cytokines was compared. The cells were preincubated with the either IL-1β, IL-3, IL-4, IL-5, IL-6, IL-10, IL-12, IFN-γ, TNFα, GM-CSF, FAS-L or IGF-1 overnight, and then exposed to 100 ng/ml LeTx. The only cytokines to demonstrate an increased cell viability over that in cells treated with no cytokines were IL-4 and IL-6. Therefore, IL-4 and IL-6 provide some protection against the toxic effects of LeTx.

### Example 8

### LeTx Causes Apoptotic Changes in Monocyte-Derived Macrophages

A high level of systemic bacteremia is a typical feature of anthrax. To study pathogenecity of B. anthracis on human blood cells, the effect of LeTx on freshly isolated PBMCs and monocytes was tested. The PBMCs and monocytes were resistant to lysis in presence of LeTx, in contrast to mouse RAW 264.7 cells. At the highest concentration of LeTx tested (500 ng/ml LF and 500 ng/ml PA), no lysis or changes in fluorescence staining patterns (see below) were detected after 15 to 24 h incubation (data not shown).

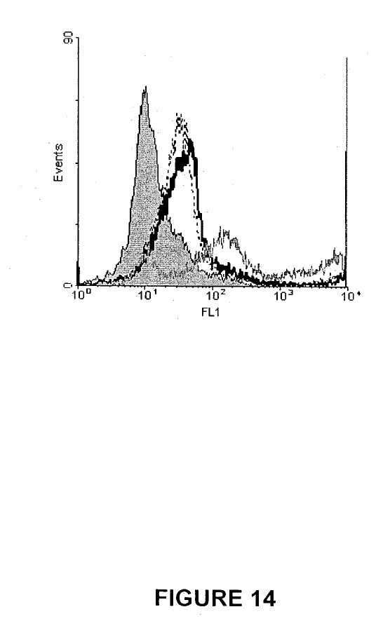

LeTx-induced nucleosomal fragmentation, typical for apoptosis (12), was also detected using the Tunel assay in FIG. 14. In FIG. 14, cells were preincubated with IFN-γ (100 u/ml) for 48 h and treated with different concentrations of toxin for 15 h in culture media. Staining was measured by flow cytometry. The treatments are as follows: dotted line, PA (500 ng/ml); thick solid line, PA (500 ng/ml)+LF (500 ng/ml); thin solid line, staurosporine (50 μM) as positive control. The gray area represents untreated control cells. Fluorescence in the green channel was recorded. These results, combined, demonstrate a role of LeTx in causing apoptosis or anthrax-infected human cells.

### Example 9

### Lethal Toxin Suppresses the Innate Immune Response and Inhibits Bactericidal Activity of PBMCs

Bacterial infection typically induces a proinflammatory response of PBMCs and other immune cells as a result of the innate signaling involving toll-like receptors that recognize a variety of antigens from gram-negative and gram-positive bacteria. (Akira et al. 2001.) One of the pathogenic functions of LeTx can be the suppression of the innate response of monocytes/macrophages and, perhaps, other cell types of PBMCs. The release of proinflammatory cytokines in LeTx-treated PBMCs by surface antigens of anthrax bacillus, such as cell wall components (CW), can be used as a sensitive indicator of this process.

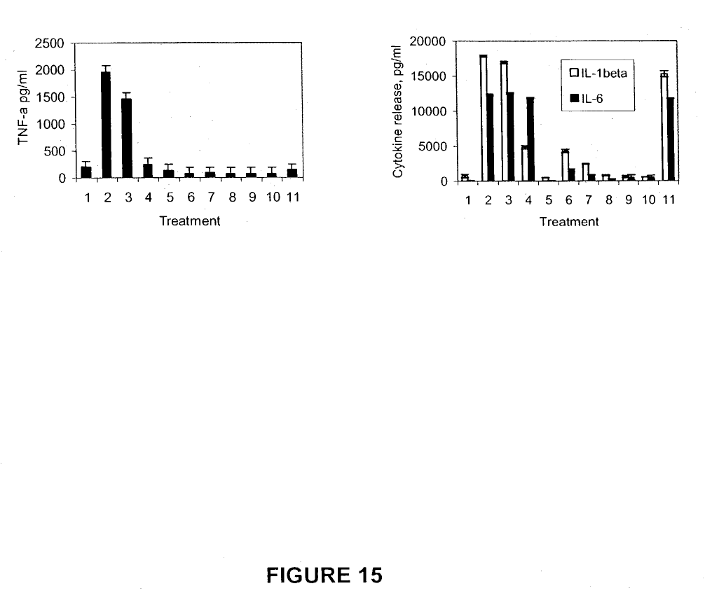

In FIG. 15, cytokine release, detected by ELISA after 48 h stimulation, is demonstrated. Treatments are as follows: cells only control (1), cell wall (CW), 1 μg/ml (2), CW, 0.5 μg/ml (3), CW, 0.1 μg/ml (4), LF, 0.5 μg/ml+PA, 0.1 μg/ml (5), CW, 1 μg/ml+LF, 0.5 μg/ml+PA, 0.1 μg/ml (6), CW, 0.5 μg/ml+LF, 0.5 μg/ml+PA, 0.1 μg/ml (7), CW, 0.1 μg/ml+LF, 0.5 μg/ml+PA, 0.1 μg/ml (8), LF, 0.5 μg/ml (9), PA, 0.1 μg/ml (10), LPS, 10 ng/ml (11).

Indeed, PBMCs stimulated with anthrax CW responded by strongly increasing in the production of IL-1β, TNF-α and IL-6, typical for the innate immune response (FIG. 15), whereas LeTx treatment effectively abrogated the induction of the cytokine release. Similar effects were seen with isolated MDMs (data not shown). Therefore, LeTx acts by inhibiting the production of proinflammatory cytokines in the innate immune response.

### Example 10

### Anthrax Infection in MDMs Results in Apoptosis-Positive Cells

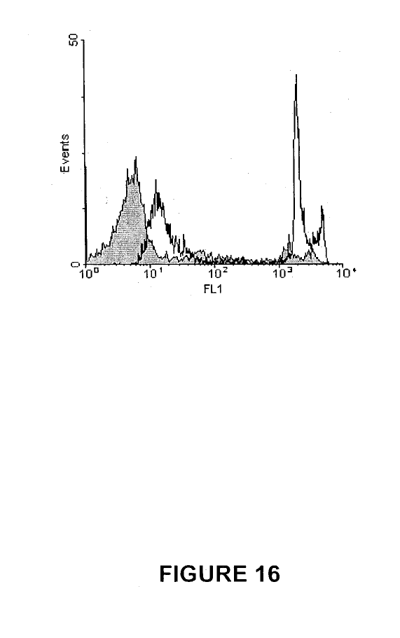

The apoptotic morphology of human MDMs treated with LeTx, combined with the observation that the LF, EF, and PA genes are expressed early in the infection process (Guidi-Rontani et al. 1999), suggested that apoptosis can be detected in spore-infected blood cells. FIG. 16 shows a histogram of green fluorescence of PBMCs in flow cytometry experiment 24 h after infection with anthrax (Sterne) spores. PBMCs we gated using a scatter plot and the spore:cell ratio was 10:1. Signals from growing bacterial cells do not overlap with signals from apoptotic cells.

In cells stained with apoptotic dyes at different time points after the addition of spores, gating of flow cytometry scatter plots allows almost complete separation of signals from spores/vegetative bacilli and infected cells on fluorescence plots. Within 24 h of infection, the cell population corresponding to uninfected macrophages disappeared, while the intensity of cells staining positively for apoptosis-like membrane permeability changes increased considerably. This confirms the role of apoptosis in anthrax infection, as mediated by LeTx.

### Example 11

### Apoptosis Array Experiments With LeTx-Treated Cells

Expression arrays provide rapid semiquantitative tools to identify differentially expressed genes in response to stimuli and treatments. Several analyses of host-pathogen interactions have already been reported, but none in the area of anthrax pathogenesis (for review see Manger et al., 2000; Cummings and Relman, 2000; Boldrick et al., 2002; Nau et al., 2002).

In vitro gene expression. Previously, it was shown that LeTx induces changes in murine and human cells that are typical of apoptosis. Taken together it is clear that one of the known apoptosis pathway is involved in LeTx-induced cell death in vitro and its role in anthrax pathogenesis needs to be determined. By analyzing host gene expression responses both in vitro and in vivo, the following are demonstrated: 1) the genes of the apoptosis pathway that are involved in host immune response to B. anthracis pathogen in vitro and murine in vivo model, 2) the role of LeTx in anthrax pathogenesis; and 3) the features of the host response to B. anthracis that are common to both in vitro and in vivo models.

cDNA arrays are designed that represent approximately 400 human and mouse genes involved in apoptotic events, such as cell cycle regulators, caspases, signal transduction factors, cytokines and their receptors, and other immunomodulating factors, in addition to the housekeeping genes, and negative controls. Because macrophages are known to play key roles in host defense by recognizing, engulfing, and killing bacteria, they are the focus. Apoptotic transcriptional events are detected in LeTx-treated, B. anthracis (Sterne) and delta-Sterne (pXO1⁻) infected murine macrophages, human PBMC and human PMM versus untreated cells.

Briefly, murine macrophages (RAW 264.7) and human PMM are cultured in 24 well plates (1×10⁶ cells/ml) and plated in serum-free media stimulated with IFN-γ (10 U/ml) overnight, and treated with LeTx (between 1 and 100 ng/ml with a constant PA of 500 ng/ml). At several intervals (after 1 h, 2 h, and 3 h for RAW cells and 24 h for human cells) cells are lysed and whole RNA is isolated using the Trizol LS method (Bloch et al., 1991). RT-PCR is performed, and cDNA is produced using protocol from Cyscribe cDNA Post Labelling kit (Amersham-Pharmacia Biotech). The printed microarray slides are rehydrated for 90 sec by suspension over 100 μl of 3×SSC in a small chamber. The slides are snap-dried by placing on a heating block for 4 sec. DNA is crosslinked to the coated slide by UV irradiating at 650 μJ. The microarray slides is prehybridized for 45 min at 45° C. in prehybridization buffer (1×SSC, 0.1% SDS, and 1% BSA), washed extensively with water, dipped into isopropanol, and air dried. The fluorescent-tagged probe in hybridization buffer (25% formamide, 5×SSC, 0.1% SDS) is heated at 95° C. for three min and placed on ice. A cover slip is placed over the microarray, and the probe applied using capillary action to draw the solution under the coverslip. The slide is placed in a hybridization chamber (Corning) and 10 μl of 3×SSC is added to the humidity wells. The chamber is sealed and incubated at 45° C. overnight. Slides are washed twice for 10 min each time at 45° C. with shaking in: 1×SSC, 0.2% SDS; 0.1×SSC, 0.1% SDS and finally 0.1×SSC. The slides are then briefly rinsed with water and centrifuged for 6 min at 500 rpm to dry.

Murine and human cells are infected with B. anthracis spores (at MOI from 1 to 10) to demonstrate similarities in gene expression between LeTx treated and anthrax-infected cells and the role of LeTx in the anthrax pathogenesis. The experimental details of cell infection with spores are similar to those described in Dixon et al., (2000) and Popov et al. (2002a). Heat-killed spores and vegetative cells (by boiling for 2 h) serve as controls to define the contribution of the infectious process, and to distinguish it from the proinflammatory response. Based on data from transcriptional profiling of macrophages and epithelial cells to Salmonella, a significant portion of inflammatory response at the level of transcription is conserved across different cell types (Eckmann et al., 2000; Rosenberger et al., 2000). These studies revealed many commonly induced genes, including leukemia inhibitory factor (LIF), MIP2a, and IRF1. Therefore, gene expression patterns of B. anthracis infection in different human cell types in vitro are demonstrated to identify common and cell type-specific responses to B. anthracis infection. Human peripheral blood monocytes neutrophils are infected with B. anthracis spores and the set of induced genes in different cell types are examined. Cells treated with conditioned media are also analyzed.

In vivo gene expression. The majority of experiments examining host-gene expression responses include measurements at only one time point. Therefore, shifts in kinetics of the response to stimuli may be interpreted as a significant difference. By looking at gene expression changes at different time points (e.g. different stages of the infection) the specific apoptosis-related genes involved at different stages of infection are demonstrated.

Different stages of anthrax infection in infected mice are examined to demonstrate the apoptosis-related genes that play a role in the early and late stages of anthrax infection. Mice sensitive to anthrax (such as C57BL/6) are infected with 46 LD₅₀ of B. anthracis (Sterne) and delta-Sterne (pXO1⁻) spores and are monitored for survival. Mice are sacrificed at several time points (12, 24 and 48 h) after infection and RNA is extracted from blood and spleens (spleen has the highest bacteremia level in Sterne-challenged mice). Mice are infected with several doses of B. anthracis (Sterne) and delta-Sterne (pXO1⁻) spores (between 10³ and 10⁷ spores/mouse) to show that dose plays a role in a pattern of gene expression. In addition, the routes of administration of the pathogen are important. B. anthracis spores are administered intranasally, to mimic aerosol attack, and intraperitoneally, to mimic systemic immune response. In the first case, the lungs are also studied along with the spleen.

Because the infectious dose of the pathogen and route of the infection affects gene expression, the dose effect of B. anthracis in murine model of infection is demonstrated. Mice are infected with several doses of B. anthracis (Sterne) and delta-Sterne (pXO1⁻) spores (between 10³ and 10⁷ spores/mouse) to show that dose affects the pattern of gene expression. In addition, the route of administration of the pathogen effects the pattern of gene expression. B. anthracis spores are administered intranasally, mimicking aerosol attack, and intraperitoneally, mimicking systemic immune response. In the first case, lungs are studied as test organ along with spleen.

### Example 12

### Inhibition of Toxin Intracellular Activity

Evaluation of the protective activity of broad-spectrum caspase inhibitor (benzyloxycarbonyl-Val-Ala-Asp-fluoromethylketone; z-VAD-fmk, z-VAD) in murine model of anthrax using different doses (10 to 50 mg/kg, ip) and administration schedules demonstrates a new approach to inhibiting anthrax toxin. The inhibitor treatment starts 2 days before challenge, at the time of challenge, and 1, 2, 3, and 4 days after 3 to 10 LD₅₀ spore challenge (ip). B. anthracis (Sterne) strain are used. Anthrax-sensitive strains of mice, such as C57/B16 or A/J, are treated every 12 h for several days (up to 10 days). Survival of animals is monitored for three weeks.

In addition, the effects of a caspase-3 inhibitor (benzyloxycarbonyl-Asp-Glu-Val-Asp-chloromethylketone; z-DEVD.cmk), and a caspase-1 inhibitor (acetyl-Tyr-Val-Ala-Asp-chloromethylketone; Ac-YVAD.fmk) are evaluated. These inhibitors have proven effective in preventing death in a well-characterized murine model of TNF-induced apoptosis in vivo (Jaeschke et al., 1998), endotoxin-induced myocardial dysfunction (Neviere et al., 2001), and apoptosis-induced by Legionella pneumophila during the early stages of infection. The efficacy of the inhibitors is correlated with their protective capacity in cell culture, as well as with bacterial load in spleen determined at days 1 through 5, and finally at day 14. RAW264.7 cells are treated with LeTx (between 1 and 100 ng/ml) and a range of inhibitor concentrations (1 to 100 μM). After 4 h the viability of cells are assessed using the MTT assay or flow cytometry. Spleens are removed at each time point, weighed, and each homogenized in 2 ml of sterile PBS with 0.1% Triton. Homogenate (100 μl) and further dilutions are plated, after rigorous vortexing for 30 seconds, on blood agar plates to quantitate bacteria as described before (Dixon et al., 2000).

Inhibitors of matrix metalloproteinases (MMPs) are used to demonstrate that surface shedding of transmembrane FasL contributes to LeTx activity. The MMP and caspase pathways have several areas of overlap with the cytokine network. Inflammatory cytokines or growth factors can regulate the expression of MMPs, while cytokine activation of cells can lead to increased processing of MMPs from inactive to active forms, modulation of caspases, and ultimately to modulation of apoptosis.

### Example 13

### Survival of Inhibitor Treated A/J Mice Infected With 5×10⁵ CFU of B. anthracis Spores

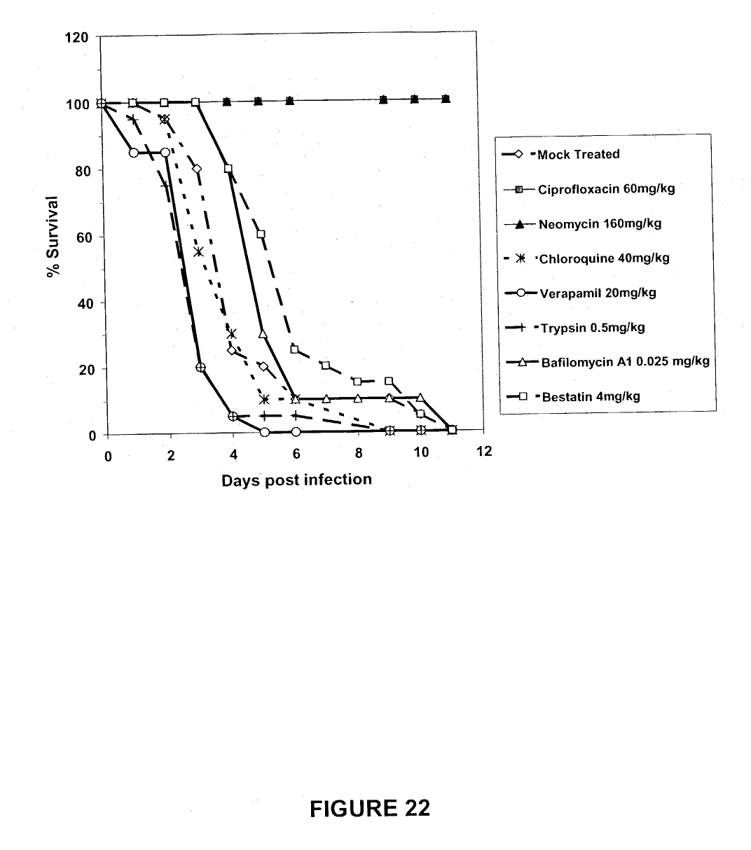

Survival of inhibitor treated A/J mice infected with 5×10⁵ CFU of B. anthracis spores was demonstrated. Mice were treated at days −1, 0, 1 and 4 with ciprofloxacin (60 mg/kg), doxycycline (160 mg/kg), neomycin (160 mg/kg), chloroquine (40 mg/kg), verapamil (20 mg/kg), trypsin (0.5 mg/kg), bafilomycin A1 (0.025 mg/kg), bestatin (4 mg/kg), Z-VAD-fmk (20 mg/kg), or with a mock treatment. As shown in FIG. 22, all mice except those treated with ciprofloxacin or neomycin were dead by 11 days. In contrast, 100% of the mice treated with ciprofloxacin and neomycin were alive after 11 days.

### Example 14

### GM-CSF Administration Increases Survival of B. anthracis (Sterne) Infected Mice

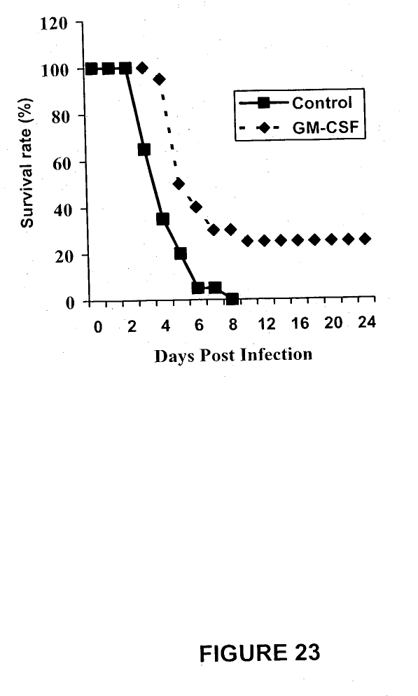

A/J mice were injected intraperitoneally with 5×10⁵ spores per mice (approximately 4×LD50) on day 0. mGM-CSF was administered intranasally on days −2, 0, +2, +4 at the dose of 2×10⁴ units/mouse/day. FIG. 23 shows that 20% of the mice treated with GM-CSF survived after 24 days, while none of the control mice survived after 8 days.

### Example 15

### Pre-exposure zVAD-fmk Treatment Increases Survival of B. anthracis (Sterne)-Infected Mice

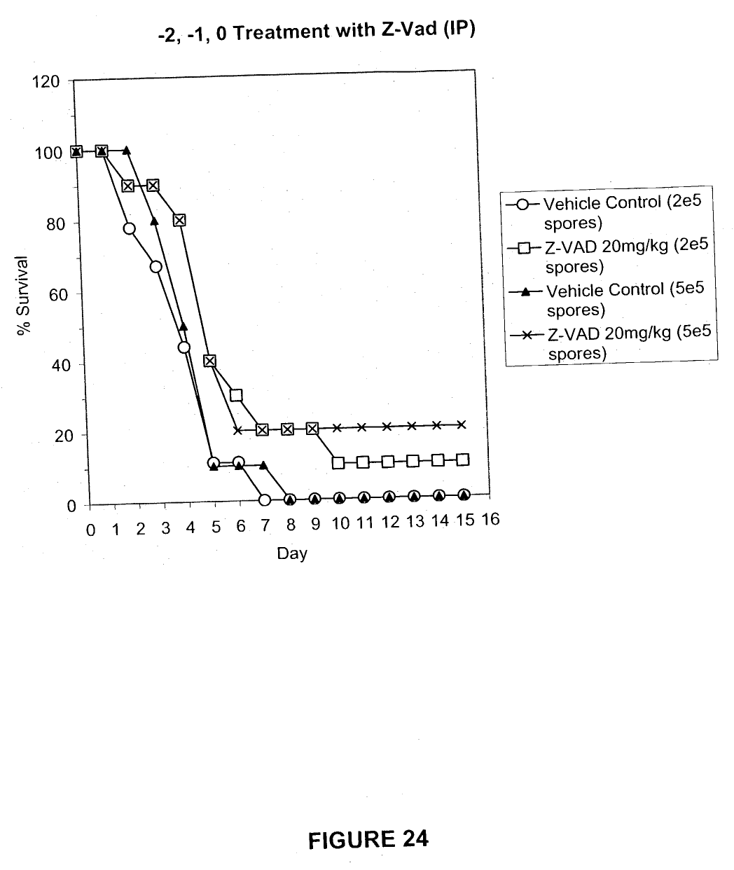

A/J mice were injected peritoneally with 2×10⁵ and 5×10⁵ spores per mice (approximately 2× and 4×LD50) on day 0. zVAD-fmk was administered intraperitoneally on days −2, −1, and 0 at the dose of 20 mg/mouse/day. As shown in FIG. 24, all of the control mice were dead by day 8, whereas 20% of the mice treated with zVAD-fmk survived by day 15 when infected with 5×10⁵ spores and 10% of the mice survived after infection with 2×10⁵ spores.

### Example 16

### Post-Exposure zVAD-fmk Treatment Increases Survival of B. anthracis (Sterne)-Infected Mice

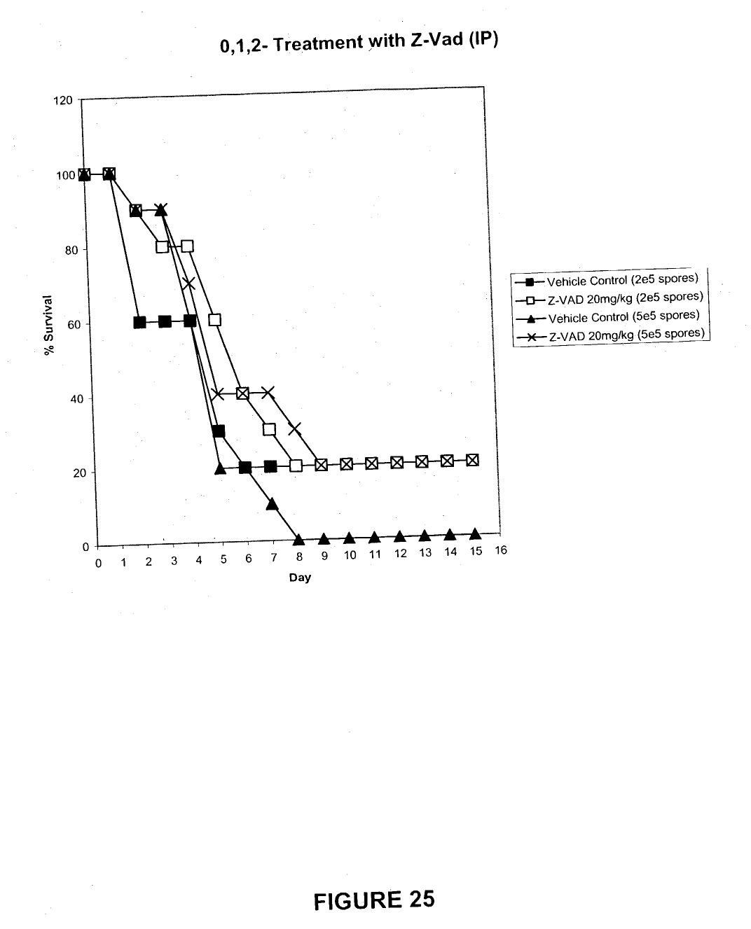

A/J mice were injected peritoneally with 2×10⁵ and 5×10⁵ spores per mice (approximately 2× and 4×LD50) on day 0. zVAD-fmk was administered intraperitoneally on days 0, 1, and 2 at the dose of 20 mg/mouse/day. FIG. 25 demonstrates that all of the control mice infected with 5×10⁵ spores were dead by day 8, while 20% of those treated with z-VAD-fmk were alive by day 15.

### Example 17

### Protection of B. anthracis-Infected Mice With a Combined Early and Late Administration of zVAD and Bestatin

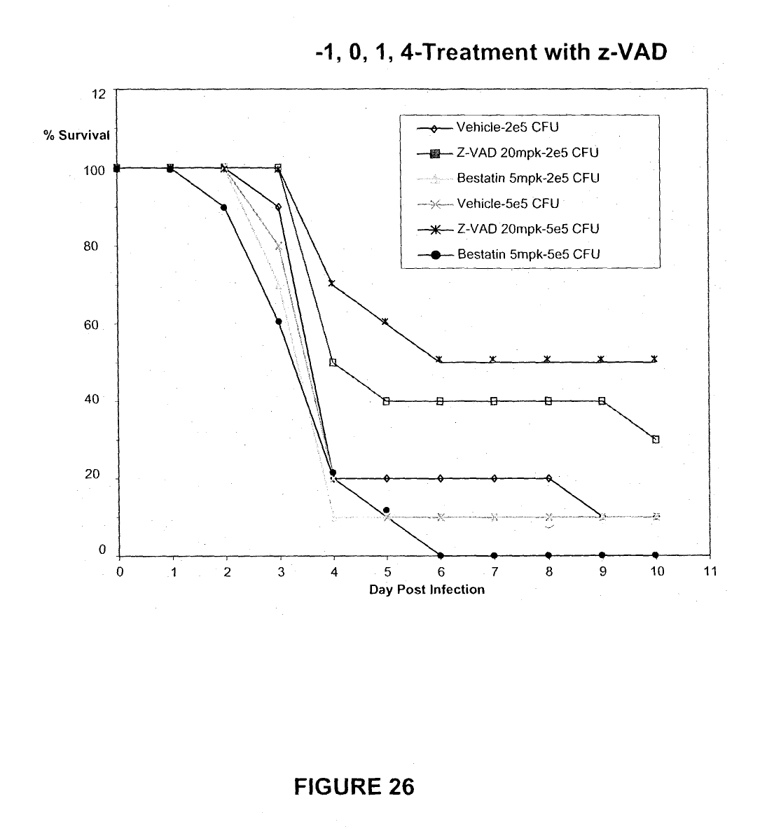

A/J mice were injected peritoneally with 2×10⁵ and 5×10⁵ spores per mice (approximately 2×and 4×LD50) on day 0. zVAD-fmk and bestatin (at the dose of 5 mg/mouse/day) were administered intraperitoneally on days −1, 0, 1, and 4. FIG. 26 shows that with all treatments only 50% or fewer mice were alive after 6 days.

### Example 18

### Protection of B. anthracis-Infected Mice With the Administration of Neomycin Combined With the Late Administration of Ciprofloxacin

DBA mice were injected intraperitoneally with approximately LD80 of spores on day 0. Neomycin (at the dose of 1 and 5 mg/kg/day) was administered intraperitoneally daily on days −1 till day 11. At day 0 mice were challenged with the indicated amounts of anthrax (Sterne) spores. A delayed antibiotic treatment (ciprofloxacin, 50 mg/kg/day) was started at day 2, and continued until day 11. Mortality was recorded until day 15. As shown in FIG. 27, treatment with ciprofloxacin alone resulted in only 50% survival of mice by day 12. Neomycin alone at either 1 mg/kg or 5 mg/kg resulted in survival of less than 20% of the mice after 10 days. Unexpectedly, when neomycin was administered at 5 mg/kg before ciprofloxacin, 100% of the mice survived at least 14 days and greater than 80% of the mice survived after neomycin was administered at 1 mg/kg.

### Example 19

### Protection of B. anthracis-Infected Mice With the Administration of Bestatin Combined With the Late Administration of Ciprofloxacin

DBA mice were injected intraperitoneally with approximately LD80 of spores on day 0. Bestatin (at the dose of 5 and 25 mg/mouse/day) was administered intraperitoneally daily on days −1 until day 11. At day 0, mice were challenged with the indicated amounts of anthrax (Sterne) spores. A delayed antibiotic treatment (ciprofloxacin, 50 mg/kg/day) was started at day 2, and continued until day 11. Mortality was recorded until day 15. As shown in FIG. 28, more than 30% of the mice were dead by day 9 when treated with bestatin alone or not treated. When treated with ciprofloxacin alone, approximately 50% of the mice were alive after 15 days. Unexpectedly, 100% of the mice treated with both bestatin at 5 mg/kg and ciprofloxacin were alive after 15 days, and approximately 90% of the mice treated with bestatin at 25 mg/kg and ciprofloxacin were alive after 15 days.

### Example 20

### Protection of B. anthracis-Infected Mice With the Administration of Ciprofloxacin

A/J mice were injected intraperitoneally with approximately LD80 of spores on day 0. Treatment with 60 mg/kg/day of ciprofloxacin was done for 5 days starting on 1, 2, or 3 days post infection, or the day prior to or the day of infection. As shown in FIG. 29, 100% of the mice survived at least 14 days when the day of infection, the day prior to infection, or one day after infection. Only 70% of the mice survived 5 days when treated two days after infection, and 20% or fewer mice survived 5 days when treated three days after infection or were not treated.

### Example 21

### Modulation of Cell Survival and Apoptosis With GM-CSF in Presence of LeTx

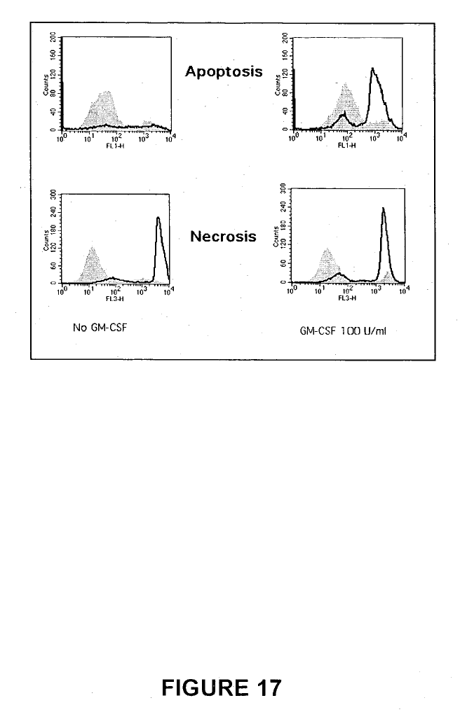

Because cytokines can modulate apoptosis, different cytokines may differentially influence the outcome of LeTx activity in cell. To demonstrate this Raw 264.7 cell susceptibility to LeTx is assayed in the presence of the murine cytokines GM-CSF, tumor necrosis factor alpha (TNF-α), interleukin-2 (IL-2), interleukin-12 (IL-12) and IFN-γ. The lytic effect of the toxin was monitored by flow cytometry by counting a number of unlysed cells by forward scatter for a certain period of time. IL-2 and IL-12 were without effect. Murine TNF-α, known to be a cytolytic and proapoptotic cytokine, greatly increased cell lysis by LeTx at all concentrations tested. Murine IFN-γ also increased cell susceptibility to lysis, in agreement with its property to sensitize cells to apoptosis. In contrast, in presence of LeTx, GM-CSF increased cell survival. Without GM-CSF pretreatment, after exposure to 32 ng/ml LeTx, the majority of cells undergo lysis (FIG. 2, left top panel), and only dead cells could be counted by flow cytometry (FIG. 17, left bottom panel). In contrast, in presence of GM-CSF, the cells become protected from lysis, which resulted in the appearance of an intensive peak corresponding to viable cells (FIG. 17, right top panel). This conclusion is further illustrated in FIG. 5 where relative number of cells in the apoptotic stage versus dead and alive cells has been calculated in quadrants of dot-plots in a similar experiment at 16 ng/ml LeTx. These results show that GM-CSF interferes with the toxin pathway slowing down the progression of cellular events from apoptosis to lysis. It is therefore reasonable to expect a protective effect of GM-CSF directed against intoxication and lysis of macrophages in the course of anthrax infection.

### Example 22

### GM-CSF Administration Increases Survival of Anthrax-Infected Mice

The invention encompasses GM-CSF administration as a treatment for anthrax. In the following techniques provided by the invention, the LD50 of Bacillus anthracis (Sterne) spores is established for a particular batch of spores which are used throughout the whole study. In these techniques, infected mice are treated with GM-CSF using different regiments. Each study lasts 2 to 3 weeks. All in vivo experiments will be carried out using male C57/6 mice (4 to 5 weeks old). Reduction of bacterial burden after treatment with recombinant murine GM-CSF

Mice (n=6 to 10 in each group) are challenged intraperitoneally (i.p.) with 1×, 2×, and 4×LD50 spores and treated with 50 μg of GM-CSF/kg/day i.p., simultaneously with initiation of infection. The dosage and timing are selected based on previous studies in the literature (Bermudez et al., 1994; Mandujano et al., 1995; Deepe and Gibbons, 2000). The mean number of colony-forming units (CFU) in spleens of GM-CSF-treated mice is determined compared to infected controls without GM-CSF treatment. Spleen is the most indicative of bacterial load organ in B. anthracis (Sterne) infected mice (T.Voss, Southern Research Institute, personal communication). The bacterial burden is assessed each day within the first week, and then at the end of the second weeks to determine if continued treatment with rmGM-CSF would modify bacterial recovery beyond week 1.

Dose Response Profile of GM-CSF

Mice are treated with murine GM-CSF at 0.5, 5, or 50 μg/kg/day (4×10⁴, 4×10⁵, or 4×10⁶ U/kg/day) simultaneously with initiation of infection. One week later, mice are sacrificed and tested for the number of bacterial CFU. Pretreatment with rmGM-CSF and enhancment of the host's anti-anthrax activity

Mice are treated with GM-CSF beginning 2 days before infection, as well as 0 and 1 day after infection, and the impact of cytokine treatment is assessed. The dosage of GM-CSF is adjusted, if necessary, based on the results, above. Statistical differences between different regiments of pretreatment are evaluated. Assays are repeated, if necessary, to substantiate the efficacy of pretreatment. Ex vivo influence of GM-CSF on anti-anthrax activity of peritoneal macrophages

To determine that treatment with GM-CSF directly arms peritoneal macrophages to inhibit the growth of B. anthracis, mice are treated using the protocol in the techniques described above which provides the best protective effect. Peritoneal macrophages from infected mice are recovered with phosphate-buffered saline in pools (per treatment group) and put in culture at 10⁶ cell/well in 96-well plates using DMEM media supplemented with 10% fetal calf serum in the absence of antibiotics and phenol red. The next day the monolayers of cells are infected with anthrax spores at concentrations, 10⁵, 10⁶ and 10⁷ spores/well. After incubation for 3 h cells are lysed with 1% saponin, and the amount of survived spores is determined using Alamar Blue® technique according to the manufacturer's instructions (Biosource International, USA). This fluorescence detection system is routinely used for determination of the number of viable anthrax spores.

Bacillus anthracis (strain Sterne) spores are prepared by inoculating liquid LB broth. After a ratio of spore to vegetative bacteria reaches 99:1, the spores are pelleted, washed five times with distilled water, and the concentration are adjusted to 1×10⁹ spores/ml. Animals are infected using intraperitoneal injections of spores. This way of challenge is considered as a model of intralymphatic spore germination and multiplication similar to that in inhalational anthrax.

Bacterial burden is expressed as mean colony forming units (CFU) per gram of organ±standard error. Commercially purchased murine recombinant GM-CSF (with specific activity specific activity about 7×107 U/mg) are diluted in phosphate-buffered saline (pH 7.4) containing 1% bovine serum, and administered to mice on a daily basis. No toxicity has been observed in the literature in mice given 50 μg/kg/day (4×10⁶ U/kg/day) i.p. for 21 days (Deepe and Gibbons, 2000).

The log rank test is used for statistical analyses of differences in survival. Student's t-test is employed to analyze differences in bacterial burden of organs. If the data are not normally distributed, the Mann-Whitney test is used.

### References

The following references are cited herein. The entire disclosure of each reference is relied upon and incorporated by reference herein.

1. Ahn J H, Park S M, Cho H S, Lee M S, Yoon J B, Vilcek J, Lee TH. J (2001) Non-apoptotic signaling pathways activated by soluble Fas ligand in serum-starved human fibroblasts. Mitogen-activated protein kinases and NF-kappaB-dependent gene expression. Biol Chem 2001 276: 47100-6.

2. Akira, S., Takeda, K. and Kaisho, T. (2001) Nature Immunol. 2: 675-680.

3. Angel J B, High K, Rhame F, Brand D, Whitmore J B, Agosti J M, Gilbert M J, Deresinski S. Phase III study of granulocyte-macrophage colony-stimulating factor in advanced HIV disease: effect on infections, CD4 cell counts and HIV suppression. Leukine/HIV Study Group. AIDS 14: 387-95.

4. Ankarcrona M, Dypbukt J M, Bonfoco E, Zhivotovsky B, Orrenius S, Lipton S A, Nicotera P (1995) Glutamate-induced neuronal death: a succession of necrosis or apoptosis depending on mitochondrial function. Neuron 1995 15:961-73.

5. Armitage J O. (1998) Emerging Applications of Recombinant Granulocyte-Macrophage Colony-Stimulating Factor. J Am Soc Hem 92: 4491-4508.

6. Ashkenazi A, Dixit V M (1998) Death receptors: signaling and modulation. Science 281: 1305-8.

7. Bermudez L E, Martinelli J, Petrofsky M, Kolonoski P, Young L S. (1994) Recombinant granulocyte-macrophage colony-stimulating factor enhances the effects of antibiotics against Mycobacterium avium complex infection in the beige mouse model. J Infect Dis 169(3):575-80.

8. Bhatnagar R, Ahuja N, Goila R, Batra S, Waheed S M, and Gupta P. (1999) Activation of phospholipase C and protein kinase C is required for expression of anthrax lethal toxin cytotoxicity in J774A.1 cells. Cell Signal 11(2): 111-6.

9. Bicknell G R, Cohen G M (1995) Cleavage of DNA to large kilobase pair fragments occurs in some forms of necrosis as well as apoptosis. Biochem Biophys Res Commun 207: 40-7.

10. Bingisser R, Stey C, Weller M, Groscurth P, Russi E, Frei K (1996) Apoptosis in human alveolar macrophages is induced by endotoxin and is modulated by cytokines. Am J Respir Cell Mol Biol 15: 64-70.

11. Boldrick J C, Alizadeh A A, Diehn M, Dudoit S, Liu C L, Belcher C E, Botstein D, Staudt L M, Brown P O, Relman D A. (2002) Stereotyped and specific gene expression programs in human innate immune responses to bacteria. Proc Natl Acad Sci U S A 99:972-7.

12. Bonfoco E, Krainc D, Ankarcrona M, Nicotera P, Lipton S A (1995) Apoptosis and necrosis: two distinct events induced, respectively, by mild and intense insults with N-methyl-D-aspartate or nitric oxide/superoxide in cortical cell cultures. Proc Natl Acad Sci U S A 92: 7162-6.

13. Bowman E J, Siebers A, Altendorf K. (1988) Bafilomycins: a class of inhibitors of membrane ATPases from microorganisms, animal cells, and plant cells. Proc Natl Acad Sci USA 85(21): 7972-6.

14. Bradley K A, Mogridge J. Mourez M, Collier R J, Young J A (2001) Identification of the cellular receptor for anthrax toxin. Nature 414: 225-9.

15. Braun et al. (1999) Neuroprotection by a caspase inhibitor in acute bacterial meningitis. Nature Med., 5: 298-302.

16. Brown S B and Savill J. (1999) Phagocytosis triggers macrophage release of Fas ligand and induces apoptosis of bystander leukocytes. J. Immunol. 162: 480-85.

17. Burgasov P N, editor. Sibirskaya yazva. Moscow: Meditsina; 1980.

18. Chiarugi A, Calvani M, Meli E, Traggiai E, Moroni F. (2001) Synthesis and release of neurotoxic kynurenine metabolites by human monocyte-derived macrophages. J Neuroimmunol 120(1-2):190-8.

19. Cianfrocca M, Cooley T P, Lee J Y, Rudek M A, Scadden D T, Ratner L, Pluda J M, Figg W D, Krown S E, Dezube B J. (2002) Matrix metalloproteinase inhibitor COL-3 in the treatment of AIDS-related Kaposi's sarcoma: a phase I AIDS malignancy consortium study. J Clin Oncol 20(1):153-9.

20. Cohen G M, Sun X M, Fearnhead H, MacFarlane M, Brown D G, Snowden R T, Dinsdale D (1994) Formation of large molecular weight fragments of DNA is a key committed step of apoptosis in thymocytes. J Immunol 153: 507-16.

21. Coligan, E et al. eds., Current Protocols in Immunology, John Wiley & Sons, Inc., 1999.

22. Cordeiro C, Wiseman D J, Lutwyche P, Uh M, Evans J C, Finlay B B, Webb M S. (2000) Antibacterial efficacy of gentamicin encapsulated in pH-sensitive liposomes against an in vivo Salmonella enterica serovar typhimurium intracellular infection model. Antimicrob Agents Chemother 44: 533-9.

23. Cummings C A, Relman D A. (2000) Using DNA microarrays to study host-microbe interactions.Emerg Infect Dis 6(5):513-25.

24. Deepe Jr G S and Gibbons R (2000) Recombinant Murine Granulocyte-Macrophage Colony-Stimulating Factor Modulates the Course of Pulmonary Histoplasmosis in Immunocompetent and Immunodeficient Mice. Antimicrob Agents and Chemother 3328-3336.

25. Dierdorf R, Kreuter U, Jones T C. (1997) A Role for Granulocyte-Macrophage Colony-Stimulating Factor (GM-CSF) in the Treatment of Neutropenic Patients with Pneumonia. Braz J Infect Dis 1: 68-76.

26. Dixon T C, Fadl A A, Koehler T M, Swanson J A, Hanna P C. (2000) Early Bacillus anthracis-macrophage interactions: intracellular survival and escape. Cell Microbiol 2(6):453-63.

27. Doskeland S O (1999) Ultrarapid caspase-3 dependent apoptosis induction by serine/threonine phosphatase inhibitors. Cell Death Differ 6: 1099-108.

28. Drose S, Boddien C, Gassel M, Ingenhorst G, Zeeck A, Altendorf K (2001) Semisynthetic derivatives of concanamycin A and C, as inhibitors of V- and P-type ATPases: structure-activity investigations and development of photoaffinity probes. Biochemistry 40: 2816-25.

29. Drummond D C, Zignani M, Leroux J (2000) Current status of pH-sensitive liposomes in drug delivery. Prog Lipid Res 39: 409-60.

30. Duesbery N S, Vande Woude G F (1998) Anthrax lethal factor causes proteolytic inactivation of mitogen-activated protein kinase kinase. J Appl Microbiol 87: 289-93.

31. Erwin J L, DaSilva L M, Bavari S, Little S F, Freidlander, A M, and Chanh T C (2001) Macrophage-Derived Cell Lines Do Not Express Proinflammatory Cytokines after Exposure to Bacillus anthracis Lethal Toxin. Inf Immun 69: 1175-77.

32. Ezzell J W, Ivins B E, Leppla S H (1984) Immunoelectrophoretic analysis, toxicity, and kinetics of in vitro production of the protective antigen and lethal factor components of Bacillus anthracis toxin. Infect Immun 45: 761-7.

33. Fadell B, Orrenius S, Zhivotovsky B (2000) Apoptosis in human disease: a new skin for the old ceremony? Biochem Biophys Res Commun 266: 699-711.

34. Faris M, Kokot N, Latinis K, Kasibhatla S, Green D R, Koretzky G A, Nel A (1998) The c-Jun N-terminal kinase cascade plays a role in stress-induced apoptosis in Jurkat cells by up-regulating Fas ligand expression. J Immunol 160: 134-44.

35. Fedotova IuM, Ulanova A A (1970) [The specificity of anthrax toxin obtained in vitro] Zh Mikrobiol Epidemiol Immunobiol 47: 111-3.

36. Feng G. and Kaplowitz N. (2002) Am. J. Physiol. Gastrointest. Liver Physiol. 282: 825-834.

37. Fish D C, Klein F, Lincoln R E, Walker J S, Dobbs J P. (1968) Pathophysiological changes in the rat associated with anthrax toxin. J Infect Dis 118(1):114-24.

38. Flad H D, Grage-Griebenow E, Petersen F, Scheuerer B, Brandt E, Baran J, Pryjma J, Ernst M (1999) The role of cytokines in monocyte apoptosis. Pathobiology 67: 291-3.

39. Fladmark K E, Brustugun O T, Hovland R, Boe R, Gjertsen B T, Zhivotovsky B, Friedlander A M (1986) Macrophages are sensitive to anthrax lethal toxin through an acid-dependent process. J Biol Chem 261: 7123-6.

40. Gao L, Abu Kwaik Y (2000a) Hijacking of apoptotic pathwaysby bacterial pathogens. Microbes Infect 2: 1705-19.

41. Gao L Y, Kwaik Y A (2000b) The modulation of host cell apoptosis by intracellular bacterial pathogens. Trends Microbiol 8: 306-13.

42. Garcia-Calvo, et al. (1998) Inhibition of human caspases by peptide-based and macromolecular inhibitors. J. Biol. Chem. 49: 32608.

43. Grinberg L M, Abramova F A, Yampolskaya O V, Walker D H, Smith J H (2001) Quantitative pathology of inhalational anthrax I: quantitative microscopic findings. Mod Pathol 14: 482-95.

44. Groves M D, Puduvalli V K, Hess K R, Jaeckle K A, Peterson P, Yung W K, Levin V A. (2002) Phase II trial of temozolomide plus the matrix metalloproteinase inhibitor, marimastat, in recurrent and progressive glioblastoma multiform. J Clin Oncol 20:1383-8.

45. Guidi-Rontani C, Weber-Levy M, Labruyere E, and Mock M. Germination of Bacillus anthracis spores within alveolar macrophages (1999a) Mol Microbiol 31: 9-17.

46. Guidi-Rontani C, Pereira Y, Ruffie S, Sirard J C, Weber-Levy M, and Mock M. (1999b) Identification and characterization of a germination operon on the virulence plasmid pXO1 of Bacillus anthracis Mol Microbiol 33: 407-414.

47. Gulbins E, Jekle A, Ferlinz K, Grassme H, Lang F (2000) Physiology of apoptosis. Am J Physiol Renal Physiol 279: F605-15.

48. Hakem R, Hakem A, Duncan G S, Henderson J T, Woo M, Soengas M S, Elia A, de la Pompa J L, Kagi D, Khoo W, Potter J, Yoshida R, Kaufman S A, Lowe S W, Penninger J M, Mak T W (1998) Differential requirement for caspase 9 in apoptotic pathways in vivo. Cell 94: 339-52.

49. Hanna P C, Acosta D, Collier R J (1993) On the role of macrophages in anthrax. Proc Natl Acad Sci USA 90: 10198-201.

50. Hanna P C, Kruskal B, Ezekowitz R et al. (1994) Role of macrophages oxidative burst in the action of anthrax lethal toxin. Mol Med 1: 7-18.

51. Hanna P. Anthrax pathogenesis and host response (1998) Curr Top Microbiol Immunol 225:13-35.

52. Hashimoto S, Ishisaki A, Yamato K, Aiko K, Amagasa T, Nishihara T (2001) Intracellular apoptosis-inducing factor is induced by a vacuolar type H+−ATPase inhibitor in B lineage cells. J Cell Physiol 186: 65-72.

53. Hidalgo M and Eckhardt S G (2001) Development of matrix metalloproteinase inhibitors in cancer therapy. J Natl Cancer Inst 93: 178-93.

54. Hohlbaum A M, Gregory M S, Ju S T, Marshak-Rothstein A (2001) Fas ligand engagement of resident peritoneal macrophages in vivo induces apoptosis and the production of neutrophil chemotactic factors. J Immunol 167: 6217-24.

55. Hotchkiss, et al. (2000) Caspase inhibitors improve survival in sepsis. Nature Immunol. 1: 496-501.

56. Hsu N, Young L S, Bermudez L E. (1995) Response to stimulation with recombinant cytokines and synthesis of cytokines by murine intestinal macrophages infected with the Mycobacterium avium complex. Infect Immun 63: 528-33.

57. lacoangeli A, Melucci-Vigo G, Risuleo G (2000) The ionophore monensin inhibits mouse polyomavirus DNA replication and destabilizes viral early mRNAs. Biochimie 82: 35-9.

58. Inglesby T V, Henderson D A, Bartlett J G, Ascher M S, Eitzen E, Friedlander A M, Hauer J, McDade J, Osterholm M T, O'Toole T, Parker G, Perl T M, Russell P K, and Tonat K (1999) Anthrax as a biological weapon: medical and public health management. Working Group on Civilian Biodefense. JAMA 281: 1735-1745.

59. Jaeschke H, Fisher M A, Lawson J A, Simmons C A, Farhood A, Jones D A. J (1998) Activation of caspase 3 (CPP32)-like proteases is essential for TNF-alpha-induced hepatic parenchymal cell apoptosis and neutrophil-mediated necrosis in a murine endotoxin shock model. Immunol 160(7):3480-6.

60. Jendrossek V, Grassme H, Mueller I, Lang F, Gulbins E (2001) Pseudomonas aeruginosa-induced apoptosis involves mitochondria and stress-activated protein kinases. Infect Immun 69: 2675-83.

61. Jernigan J A, et al. (2001) Bioterrorism-related inhalational anthrax: the first 10 cases reported in the United States. Emerg Infect Dis 7(6): 933-44.

62. Jonas D, Walev I, Berger T, Liebetrau M, Palmer M, Bhakdi S (1994) Novel path to apoptosis: small transmembrane pores created by staphylococcal alpha-toxin in T lymphocytes evoke internucleosomal DNA degradation. Infect Immun 62: 1304-12.

63. Juo P, Kuo C J, Reynolds S E, Konz R F, Raingeaud J, Davis R J, Biemann H P, Blenis J (1997) Fas activation of the p38 mitogen-activated protein kinase signalling pathway requires ICE/CED-3 family proteases. Mol Cell Biol 17: 24-35.

64. Kamada S, Washida M, Hasegawa J, Kusano H, Funahashi Y, Tsujimoto Y (1997) Involvement of caspase-4(-like) protease in Fas-mediated apoptotic pathway. Oncogene 15: 285-90.

65. Kiener P A, Davis P M, Rankin B M, Klebanoff S J, Ledbetter J A, Starling G C, Liles W C. (1997) Human monocytic cells contain high levels of intracellular Fas ligand: rapid release following cellular activation. J Immunol 159(4):1594-8.

66. Kim K M, Lee K, Hong Y S, Park H Y. (2000) Fas-mediated apoptosis and expression of related genes in human malignant hematopoietic cells.Exp Mol Med 32(4):246-54.

67. Kline, F., Haines, B. W., Mahlandt, B. G., DeArmon, I. A. Jr. and Lincoln, R. E. (1963) J. Bacteriol. 83, 1032-1038.

68. Lee K D, Oh Y K, Portnoy D A, Swanson J A (1996) Delivery of macromolecules into cytosol using liposomes containing hemolysin from Listeria monocytogenes. J Biol Chem 271: 7249-52.

69. Le-Niculescu H, Bonfoco E, Kasuya Y, Claret F X, Green D R, Karin M (1999) Withdrawal of survival factors results in activation of the JNK pathway in neuronal cells leading to Fas ligand induction and cell death. Mol Cell Biol 19:1751-63.

70. Leppla, S. H. (1988) Methods Ezymol. 165: 103-116.

71. Leppla S H. (1982) Anthrax toxin edema factor: a bacterial adenylate cyclase that increases cyclic AMP concentrations of eukaryotic cells.Proc Natl Acad Sci U S A 79(10):3162-6.

72. Liles W C. (1997) Human monocytic cells contain high levels of intracellular Fas ligand: rapid release following cellular activation. J Immunol 159(4):1594-8.

73. Liles W C. (2001) Immunomodulatory approaches to augment phagocyte-mediated host defense for treatment of infectious diseases. Semin Respir Infect. 16(1):11-7.

74. Lin C G, Kao Y T, Liu W T, Huang H H, Chen K C, Wang T M, Lin H C (1996) Cytotoxic effects of anthrax lethal toxin on macrophage-like cell line J774A.1. Curr Microbiol 33: 4 224-7.

75. Lutwyche P, Cordeiro C, Wiseman D J, St-Louis M, Uh M, Hope M J, Webb M S, Finlay B B. (1998) Intracellular delivery and antibacterial activity of gentamicin encapsulated in pH-sensitive liposomes. Antimicrob Agents Chemother 42: 2511-20.

76. Mangan D F, Welch G R, Wahl S M. (1991) Lipopolysaccharide, tumor necrosis factor-alpha, and IL-1 beta prevent programmed cell death (apoptosis) in human peripheral blood monocytes. J Immunol 146(5):1541-6.

77. Mangan D F, Mergenhagen S E, Wahl S M. (1993) Apoptosis in human monocytes: possible role in chronic inflammatory diseases.J Periodontol 64(5 Suppl):461-6.

78. Manger I D, Relman D A. (2000) How the host ‘sees’ pathogens: global gene expression responses to infection.Curr Opin Immunol 12(2):215-8.

79. Martin C A, Panja A (2002) Cytokine regulation of human intestinal primary epithelial cell susceptibility to Fas-mediated apoptosis. Am J Physiol Gastrointest Liver Physiol 282: G92-G104.

80. Matute-Bello G, Liles W C, Steinberg K P, Kiener P A, Mongovin S, Chi E Y, Jonas M, Martin T R. (1999) Soluble Fas ligand induces epithelial cell apoptosis in humans with acute lung injury (ARDS). J Immunol 163(4):2217-25.

81. Matute-Bello G, Liles W C, Frevert C W, Nakamura M, Ballman K, Vathanaprida C, Kiener P A, Martin T R. (2001) Recombinant human Fas ligand induces alveolar epithelial cell apoptosis and lung injury in rabbits. Am J Physiol Lung Cell Mol Physiol 281(2):L328-35.

82. Menard A, Papini E, Mock M, Montecucco C (1996) The cytotoxic activity of Bacillus anthracis lethal factor is inhibited by leukotriene A4 hydrolase and metallopeptidase inhibitors. Biochem J 1996 320 (Pt 2) 687-91.

83. Morrow M, Smith L, Garner A, Sorensen J M, Von Hoff D D, Eckhardt S G. (2000) Phase I and pharmacologic study of the specific matrix metalloproteinase inhibitor BAY 12-9566 on a protracted oral daily dosing schedule in patients with solid malignancies. J Clin Oncol 18(1):178-86.

84. Munn D H, Beall A C, Song D, Wrenn R W, (1995) Throckmorton D C. Activation-induced apoptosis in human macrophages: developmental regulation of a novel cell death pathway by macrophage colony-stimulating factor and interferon gamma. J Exp Med 181(1):127-36.

85. Nau G J, Richmond J F, Schlesinger A, Jennings E G, Lander E S, Young R A. (2002) Human macrophage activation programs induced by bacterial pathogens. Proc Natl Acad Sci U S A 99(3):1503-8.

86. Nicotera P, Ankarcrona M, Bonfoco E, Orrenius S, Lipton S A (1997) Neuronal necrosis and apoptosis: two distinct events induced by exposure to glutamate or oxidative stress. Adv Neurol 72: 95-101.

87. Nicotera P, Leist M, Single B, Volbracht C (1999) Execution of apoptosis: converging or diverging pathways? Biol Chem 380: 1035-40.

88. Ossina N K, Cannas A, Powers V C, Fitzpatrick P A, Knight J D, Gilbert J R, Palmer L E, Pancetti A R, Greenberg S, Bliska J B (1999) YopJ of Yersinia spp. is sufficient to cause downregulation of multiple mitogen-activated protein kinases in eukaryotic cells. Infect Immun 67: 708-16.

89. Park, S. and Leppla, S. H. (2000) Protein Exper. Purif. 18: 293-302.

90. Pezard C, Berche P, Mock M (1991) Contribution of individual toxin components to virulence of Bacillus anthracis. Infect Immun 59: 3472-7.

91. Pellizzari R, Guidi-Rontani C, Vitale G, Mock M, and Montecucco C (1999) Anthrax lethal factor cleaves MKK3 in macrophages and inhibits the LPS/IFN-γ-induced release of NO and TNF-α. FEBS Lett 462: 199-204.

92. Pellizzari R, Guidi-Rontani C, Vitale G, Mock M, Montecucco C (2000) Lethal factor of Bacillus anthracis cleaves the N-terminus of MAPKKs: analysis of the intracellular consequences in macrophages. Int J Med Microbiol 290: 421-7.

93. Popov, S. G., Villasmil, R., Bernardi, J., Grene, E., Cardwell, J., Wu, A., Alibek, D., Bailey, C. and Alibek, K. (2002a) Biochem. Biophys. Res. Commun. 293 349-345.

94. Popov S G, Villasmil R, Bernardi J, Grene E, Cardwell J, Popova T, Wu A, Alibek D, Bailey C, and Alibek K. (2002b) Bactericidal Activity of Human Peripheral Blood Mononuclear Cells Is Inhibited by Lethal Toxin of Bacillus anthracis. FEBS Letters, submitted.

95. Penninger J M, Mak T W (1998) Differential requirement for caspase 9 in apoptotic pathways in vivo. Cell 94: 339-52.

96. Porte F, Liautard J P, Köhler S. (1999) Early acidification of phagosomes containing Brucella suis is essential for intracellular survival in murine macrophages. Infect Immun 67: 4041-7.

97. Powell W C, Fingleton B, Wilson C L, Boothby M, Matrisian L M (1999) The metalloproteinase matrilysin proteolytically generates active soluble Fas ligand and potentiates epithelial cell apoptosis. Curr Biol 9: 1441-7.

98. Rathman M, Sjaastad M D, Falkow S. (1996) Acidification of phagosomes containing Salmonella typhimurium in murine macrophages Infect Immun 64: 2765-73.

99. Rathmell J C, Thompson C B (1999) The central effectors of cell death in the immune system. Annu Rev Immunol 17: 781-828.

100. Roucou X, Antonsson B, Martinou J C. (2001) Involvement of mitochondria in apoptosis. Cardiol Clin 19: 45-55.

101. Rowinsky E K, Humphrey R, Hammond L A, Aylesworth C, Smetzer L, Hidalgo M, Cianfrocca M, Cooley T P, Lee J Y, Rudek M A, Scadden D T, Ratner L, Pluda J M, Figg W D, Krown S E, Dezube B J. (2002) Matrix metalloproteinase inhibitor COL-3 in the treatment of AIDS-related Kaposi's sarcoma: a phase I AIDS malignancy consortium study. J Clin Oncol 20(1):153-9.

102. Rowinsky E K, Humphrey R, Hammond L A, Aylesworth C, Smetzer L, Hidalgo M, Morrow M, Smith L, Garner A, Sorensen J M, Von Hoff D D, Eckhardt S G. (2000) Phase I and pharmacologic study of the specific matrix metalloproteinase inhibitor BAY 12-9566 on a protracted oral daily dosing schedule in patients with solid malignancies. J Clin Oncol 18(1):178-86.

103. Schulz et al. (1999) Caspases as treatment targets in stroke and neurodegenerative diseases. Ann. Neurol. 45: 421.

104. Sellman R, Mourez M, Collire J (2001) Dominant-negative mutants of a toxin subunit: an approach to therapy of anthrax. Science 292: 695-7.

105. Serrao K L, Fortenberry J D, Owens M L, Harris F L, Brown L A. (2001) Neutrophils induce apoptosis of lung epithelial cells via release of soluble Fas ligand. Am J Physiol Lung Cell Mol Physiol 280(2):L298-305.

106. Smith H, Keppie J, Stainley J L (1955) The chemical basis of the virulence of Bacillus anthracis. V. The specific toxin produced by B. anthracis in vivo. Br J Exp Pathol 36: 460-472.

107. Shekhtman E M, Tomei L D, Umansky S R, Kiefer M C (1997) Interferon-gamma modulates a p53-independent apoptotic pathway and apoptosis-related gene expression. J Biol Chem 272: 16351-7.

108. Shin S, Hur G H, Kim Y B, Yeon G B, Park K J, Park Y M, Lee W S (2000) Dehydroepiandrosterone and melatonin prevent Bacillus anthracis lethal toxin-induced TNF production in macrophages. Cell Biol Toxicol 16: 165-74.

109. Singh P P, Singh S. (2001) Protection of mice from malaria after co-administration of recombinant mouse granulocyte-macrophage colony-stimulating factor and methionine-enkephalin. Eur Cytokine Netw 12(3):528-36.

110. Singh Y, Leppla S H, Bhatnagar R, Friedlander A M (1989) Internalization and processing of Bacillus anthracis lethal toxin by toxin-sensitive and -resistant cells. J Biol Chem 264: 11099-102.

111. Singh Y, Klimpel K R, Goel S, Swain P K, Leppla S H (1999) Oligomerization of anthrax toxin protective antigen and binding of lethal factor during endocytic uptake into mammalian cells. Infect Immun 67: 1853-9.

112. Smith, Micheal New progress on Lou Gerhig's disease treatment. UPI Science News. http://www.terra.es/personal/lgg43347/zVAD-fmk.htm.

113. Suda T, Hashimoto H, Tanaka M, Ochi T, Nagata S (1997) Membrane Fas ligand kills human peripheral blood T lymphocytes, and soluble Fas ligand blocks the killing. J Exp Med 186: 2045-50.

114. Sweeney J F, Nguyen P K, Omann G, Hinshaw D B (1999) Granulocyte-macrophage colony-stimulating factor rescues human polymorphonuclear leukocytes from ultraviolet irradiation-accelerated apoptosis. J Surg Res 81: 108-12.

115. Tanaka M, Suda T, Yatomi T, Nakamura N, Nagata S. (1997) Lethal effect of recombinant human Fas ligand in mice pretreated with Propionibacterium acnes.J Immunol 158(5):2303-9.

116. Tang G, Leppla S H (1999) Proteasome activity is required for anthrax lethal toxin to kill macrophages. Infect Immun 67: 3055-60.

117. Tsujimoto Y, Shimizu S, Eguchi Y, Kamiike W, Matsuda H (1997) Bcl-2 and Bcl-xL block apoptosis as well as necrosis: possible involvement of common mediators in apoptotic and necrotic signal transduction pathways. Leukemia Suppl 3 380-2.

118. Tudan C, Jackson J K, Blanis L, Pelech S L, Burt H M (2000) Inhibition of TNF-alpha-induced neutrophil apoptosis by crystals of calcium pyrophosphate dihydrate is mediated by the extracellular signal-regulated kinase and phosphatidylinositol 3-kinase/Akt pathways up-stream of caspase 3. J Immunol 165: 5798-806.

119. Vazquez J A, Hidalgo J A, De Bono S. (2000) Use of sargramostim (rh-GM-CSF) as adjunctive treatment of fluconazole-refractory oropharyngeal candidiasis in patients with AIDS: a pilot study. HIV Clin Trials 1(3):23-9.

120. Vitale G, Pellizzari R, Recchi C, Napolitani G, Mock M, Montecucco C (1998) Anthrax lethal factor cleaves the N-terminus of MAPKKS and induce tyrosine/threonine phosphorylation of MAPKS in cultured macrophages. J Appl Microbiol 87: 288.

121. Walczak H, Krammer P H (2000) The CD95 (APO-1/Fas) and the TRAIL (APO-2L) apoptosis systems. Exp Cell Res 256: 58-66.

122. Walker P R, Sikorska M (1997) New aspects of the mechanism of DNA fragmentation in apoptosis. Biochem Cell Biol 75: 287-99.

123. Watters J W, Dewar K, Lehoczky J, Boyartchuk V, Dietrich W (2001) Kif1C, a kinesin-like motor protein, mediates mouse macrophage resistance to anthrax lethal factor. Current Biology 11: 1503-11.

124. Weber S M, Levitz S M, Harrison T S. (2000) Chloroquine and the fungal phagosome. Curr Opin Microbiol 3: 349-53.

125. Weinrauch Y, Zychlinsky A (1999) The induction of apoptosis by bacterial pathogens. Annu Rev Microbiol 53: 155-87.

126. Welkos, S., Keener, T. J. and Gibbs, P. (1986) Inf. Immun., 51: 795-800.

127. Wilson D J, Alessandrini A, Budd R C (1999) MEK1 activation rescues Jurkat T cells from Fas-induced apoptosis. Cell Immunol 194: 67-77.

128. Wright G G, Mandell G L. (1986) Anthrax toxin blocks priming of neutrophils by lipopolysaccharide and by muramyl dipeptide.J Exp Med 164(5):1700-9.

129. Xia Z, Dickens M, Raingeaud J, Davis R J, Greenberg M E (1995) Opposing effects of ERK and JNK-p38 MAP kinases on apoptosis. Science 270: 1326-31.

130. Yamamoto Y, Klein T W, Tomioka M, Friedman H (1997) Differential effects of granulocyte/macrophage colony-stimulating factor (GM-CSF) in enhancing macrophage resistance to Legionella pneumophila vs Candida albicans. Cell Immunol 176:1 75-81.

What is claimed is:

1. A method of treating anthrax infection comprising administering an inhibitor of LeTx activity and reducing the levels of infection by anthrax.

2. The method as claimed in claim 1, wherein the LeTx activity is proinflammatory response inhibition.

3. The method as claimed in claim 1, wherein the LeTx activity is induction of apoptosis.

4. The method as claimed in claim 1, wherein the inhibitor of LeTx activity inhibits signaling by Fas.

5. The method as claimed in claim 1, wherein the inhibitor of LeTx activity inhibits signaling by FasL.

6. The method as claimed in claim 5, wherein inhibition of signaling by FasL is inhibition of the effects of sFas L.

7. The method as claimed in claim 1, wherein the inhibitor of LeTx activity inhibits proteases of the caspase family.

8. The method as claimed in claim 7, wherein the members of the caspase family are caspase 1 (ICE), caspase 3, caspase 4 (TX/ICH-2/ICE(rel)II), or caspase 8.

9. The method as claimed in claim 7, wherein inhibitor of proteases of the caspase family is z-VAD, z-DEVD.cmk, or Ac-YVAD.fmk.

10. The method as claimed in claim 1, wherein the inhibitor of LeTx activity is an agent that protects anthrax infected cells from loss of mitochondrial transmembrane potential.

11. The method as claimed in claim 10, wherein the agent that protects anthrax infected cells from loss of mitochondrial transmembrane potential is a caspase 9 inhibitor.

12. The method as claimed in claim 1, wherein the inhibitor of LeTx activity is an agent that enhances the ERK (MAPK)-signaling pathway.

13. The method as claimed in claim 12, wherein the agent that enhances the ERK (MAPK)-signaling pathway is GM-CSF.

14. The method as claimed in claim 1, wherein the inhibitor of LeTx activity inhibits entry of LeTx into the cell.

15. The method as claimed in claim 1, wherein the inhibitor of LeTx activity is administered in a liposome or microcapsule formulation.

16. A treatment for infection by B. anthracis comprising GM-CSF or a composition comprising GM-CSF.

17. A method of treating a patient infected with B. anthracis comprising administering GM-CSF or a composition comprising GM-CSF to a patient infected with B. anthracis and reducing the level of infection by B. anthracis.

18. The method as claimed in claim 16, further comprising protecting cells infected with B. anthracis from apoptosis by administering GM-CSF or a composition comprising GM-CSF.

19. A method of treating an infectious disease comprising administering a combination of an anti-toxin substance and an antibiotic to an infected person and decreasing the level of infection, wherein the anti-toxin substance protects host cells from microbial toxin.

20. The method as claimed in claim 19, wherein the infectious disease is anthrax, plague, Ebola, or Marburg.

21. The method as claimed in claim 19, wherein the antibiotic is ciprofloxacin.

22. The method as claimed in claim 19, wherein the anti-toxin substance inhibits at least one caspase.

23. The method as claimed in claim 22, wherein the caspase is caspase 1 (ICE), caspase 2, caspase 3, caspase 4 (TX/ICH-2/ICE(rel)II), caspase 6, or caspase 8.

24. The method as claimed in claim 22, wherein the caspase inhibitor is z-VAD or bestatin.

25. The method as claimed in claim 19, wherein the anti-toxin substance is bestatin or neomycin.

26. A method of treating anthrax infection comprising administering a substance with anti-LeTx activity and reducing the levels of infection by anthrax.

27. The method as claimed in claim 19, wherein the anti-toxin substance inhibits apoptosis.

28. The method as claimed in claim 19, wherein the anti-toxin substance inhibits proteases of the caspase family.

29. The method as claimed in claim 19, wherein the antitoxin substance is Z-vad or Z-YVAD.
